(19) United States
(12) Patent Application Publication
(10) Pub. No.: US 2026/0285979 A1
(43) Pub. Date: Sep. 24, 2026

(54) COMPOSITIONS AND METHODS FOR USING ANTIGEN-SPECIFIC APOPTOTIC DNA IMMUNOTHERAPY TO PREVENT AND TREAT SIDE EFFECTS RESULTING FROM ADMINISTRATION OF IMMUNE CHECKPOINT INHIBITORS

(71) Applicant: ADITXT, INC., Mountain View, CA (US)

(72) Inventors: Shahrokh SHABAHANG, Mountain View, CA (US); Joachim-Friedrich KAPP, Berlin (DE)

(21) Appl. No.: 19/474,580

(22) PCT Filed: Apr. 12, 2024
(86) PCT No.: PCT/US2024/024412

§ 371 Date: Oct. 10, 2025

Related U.S. Application Data
(60) Provisional application No. 63/495,984, filed on Apr. 13, 2023.

(51) Int. Cl. C07K 16/28; A61K 38/00; A61K 39/00; A61P 3/10; A61P 35/00; C07K 14/47; C12N 9/88

(52) U.S. Cl. CPC C07K 16/2818; A61P 3/10; A61P 35/00; C07K 14/4747; C12N 9/88; A61K 38/00; A61K 2039/505; C07K 2317/76; C12Y 401/01015

(57) ABSTRACT

The present disclosure provides a therapeutic composition including an immune checkpoint inhibitor and an apoptotic DNA immunotherapy and methods of using such a composition to prevent or treat adverse effects resulting from administration of an immune checkpoint inhibitor.

### BACKGROUND

Autoimmune disease results from the immune response against self-antigens, while cancer develops when the immune system does not respond to malignant cells. Thus, for years, autoimmunity and cancer have been considered as two separate fields of research that do not have a lot in common. However, the discovery of immune checkpoints and the development of immune checkpoint inhibitors (ICIs) targeting, for example, programmed cell death receptor (PD-1) and cytotoxic T lymphocyte antigen 4 (CTLA-4) pathways proved that studying autoimmune diseases can be extremely helpful in the development of novel anti-cancer drugs and improved therapies. Therefore, autoimmunity and cancer may be opposite sides of the same coin. Sakowska J, Arcimowicz Ł, Jankowiak M, et al. Autoimmunity and Cancer-Two Sides of the Same Coin. Front Immunol. 2022; 13:793234. Published 2022 May 13. doi: 10.3389/fimmu.2022.793234.

It is known that using ICIs to treat cancer poses the risk of a flare up of preexistent autoimmune disease and the development of new autoimmune manifestations in patients with preexisting autoimmunity (even if such is present as a biological only manifestation, e.g., as positive auto antibodies). For this reason, patients with preexisting autoimmune manifestations have been traditionally excluded from clinical trials as it was expected to have severe autoimmune manifestations which could exceed the potential benefit from tumor control. This subgroup of cancer patients harboring autoimmune manifestations is not trivial. See Coureau M, Meert A P, Berghmans T, Grigoriu B. Efficacy and Toxicity of Immune-Checkpoint Inhibitors in Patients with Preexisting Autoimmune Disorders. Front Med (Lausanne). 2020 May 7; 7:137. doi: 10.3389/fmed.2020.00137. PMID: 32457912; PMCID: PMC7220995. For example, depending on the definition used, a large register-based analysis (SEER) including patients with lung cancer identified between 13.5% (more restrictive definition) and 24.6% (more liberal definition) of cancer patients as having an autoimmune disease of any type. Id. (internal citation removed).

Treatment of cancer with ICIs has been tied to development of Type 1 diabetes mellitus (T1DM) in some patients. T1DM is an autoimmune disease in which insulin-producing b-cells within pancreatic islets are destroyed by an autoimmune attack coordinated by autoantigen-specific polyclonal T lymphocytes that have escaped control of immune tolerance. T1DM is an irreversible immune-related adverse event that is a rare, but potentially life-threatening complication that occurs in 0.6-1.4% of patients receiving ICIs. Xuan Chen, Alison H. Affinati, Yungchun Lee, Adina F. Turcu, Norah Lynn Henry, Elena Schiopu, Angel Qin, Megan Othus, Dan Clauw, Nithya Ramnath, Lili Zhao; Immune Checkpoint Inhibitors and Risk of Type 1 Diabetes. Diabetes Care 1 May 2022; 45 (5): 1170-1176. doi.org/10.2337/dc21-2213.

The field of immunotherapeutics is addressing defective tolerance processes with immunotherapies that have vaccine-like qualities that avoid unwanted effects characteristic of broad-acting immunosuppressive therapeutics. A promising class of immunotherapies utilize the natural cell death process, apoptosis, which is a natural non-inflammatory tolerance-inducing pathway. Antigen-presenting cells (APCs), such as dendritic cells (DCs), become tolerogenic after engulfing apoptotic cells; this enables the presentation of processed apoptotic cell autoantigens (without co-stimulation) to regulatory T cells (Tregs) for stimulation or to autoreactive memory effector T cells (Teff) for inactivation.

Developing and building upon our improved understandings regarding how immune response is altered in autoimmunity and in cancer is crucial for the proper designing of novel and selective immunotherapies. New approaches to mitigate unwanted side effects resulting from use of ICIs and better adapting available existing treatments to patients with preexistent autoimmune disease and/or who develop new autoimmune manifestations in response to ICIs are needed. Specifically, use of new therapies that promote antigen-specific immune tolerance to ward off harmful autoimmune responses in particular individuals that are receiving, or may receive, ICIs for treatment of cancer could be used to prevent development of ICI-related adverse immunological side effects, or to complement, support, and maintain or prolong continuity of treatment, and/or may meaningfully reduce the potential risks and harmful side effects associated with ICI therapy.

### BRIEF DESCRIPTION OF FIGURES

FIG. 1 shows the initial study design and time course for the study described in Example 1 and Example 2. The study design changed, however, in that the test article (TA) weekly dosing (QW) was stopped at 5 weeks instead of 8 weeks. 8-week-old female NOD/ShiLtj mice (commonly called NOD mice) providing a polygenic model for autoimmune type 1 diabetes (T1D) and characterized by hyperglycemia and insulitis were obtained and acclimated for 1 week. Following random assignment to one of the 4 groups of ten (10) mice, each animal received anti-PD1 antibody or IgG as negative control starting on Day 0 (DO). Treatment using vehicle control or ADI-100 was initiated on Day 0 (DO) and administered once a week for multiple weeks. Blood glucose levels were monitored during treatment administration and for several weeks after cessation of treatment. The initial study design was scheduled to end 84 days (D84), or twelve (12) weeks following initiation of treatment on Day 0 (DO).

FIG. 2A, FIG. 2B, FIG. 2C, and FIG. 2D provide interim data in the form of absolute glucose reading measurements taken from study subjects involved in the study depicted in FIG. 1, and as described in Example 1. Mice in the control Group 1 (subject numbers 203, 204, 207, 212, 216, 220, 226, 233, 239, and 244, also referred to as 003, 004, 007, 012, 016, 020, 026, 033, 039, and 044, respectively, in later figures) (FIG. 2A) showed relatively stable blood glucose levels for the 25-day observation period, with absolute blood glucose measurements ranging between about 80 mg/dL and 160 mg/dL, while most of the mice (9/10) in vehicle Group 2 (FIG. 2B) (subject numbers 202, 208, 209, 215, 217, 218, 232, 234, 241, and 248, also referred to as 002, 008, 009, 015, 017, 018, 032, 034, 041, and 048, respectively, in later figures), which received anti-PD1 antibodies and vehicle showed overt hyperglycemia within 11 days. Most of the mice (7/10) treated with low dose ADI-100 (1 μg/μL in a ratio of BAX/msGAD of 1:2 (BAX 17 μg+msGAD 33 μg)) Group 3 (FIG. 2C) (subject numbers 201, 219, 224, 227, 229, 230, 231, 235, 237, and 243, also referred to as 002, 001, 019, 024, 027, 029, 030, 031, 035, 037, and 043, respectively, in later figures) showed hyperglycemia within the first 8 days. In contrast, 7 of 10 mice (7/10) treated with high dose ADI-100 (2 μg/μL in a ratio of BAX/msGAD of 1:2 (BAX 34 μg+msGAD 66 μg)) Group 4 (FIG. 2D) (subject numbers 206, 214, 221, 223, 225, 236, 238, 242, 246, and 247, also referred to as 006, 014, 021, 023, 025, 036, 038, 042, 046, and 047, respectively, in later figures) showed normal glycemic levels at the end of the 25-day observation period. These data support the effectiveness of ADI-100 in controlling the effect of anti-PD1 antibodies in accelerating autoimmune diabetes in NOD mice. These data also show that the effectiveness of ADI-100 is dose dependent.

FIG. 3A, FIG. 3B, FIG. 3C, and FIG. 3D provide interim data in the form of log glucose reading measurements taken from study subjects involved in the study generally depicted in FIG. 1, and as described in Example 2. Nine out of the ten (9/10) mice in the control Group 1 (FIG. 3A), showed relatively stable blood glucose levels for the 45-day observation period while most of the mice (9/10) in vehicle Group 2 (FIG. 3B), which received anti-PD1 antibodies and vehicle showed overt hyperglycemia within 11 days while one mouse (mouse 018) did not. Most of the mice (7/10) treated with low dose ADI-100 in Group 3 (FIG. 3C) also showed hyperglycemia within the first 8 days, while some mice (3/10) showed normal glycemic levels for the 45-day observation period. In contrast, 7 of 10 mice (7/10) treated with high dose ADI-100 in Group 4 (FIG. 3D) showed normal glycemic levels during the 45-day observation period. These data support the effectiveness of ADI-100 in controlling the effect of anti-PD1 antibodies in accelerating autoimmune diabetes in NOD mice. These data also show that the effectiveness of ADI-100 is dose dependent and may be prolonged over at least six (6) weeks.

FIG. 4A, FIG. 4B, FIG. 4C, and FIG. 4D provide interim data in the form of absolute glucose reading measurements taken from study subjects involved in the study generally depicted in FIG. 1, and as described in Example 2 and corresponding to FIGS. 3A, FIG. 3B, FIG. 3C, and FIG. 3D.

FIG. 5 shows the revised and extended study design and time course to investigate the enduring effects of ADI-100 tolerance and also the effectiveness of resumed treatment to recover any mice showing evidence of lost tolerance for the study depicted in FIG. 1 and described in Example 1 and Example 2. This extension study and the results thereof are further described in Example 3. The change in the study design is indicated at day 29 (D29) to indicate that dosing was paused for all groups after the 5th dose on day 28 (D28) instead of occurring weekly for eight (8) weeks and the study ending on day 84 (D84). As shown in FIG. 5, only the remaining control Group 1 mice and the remaining high dose ADI-100 Group 4 mice continued into the Phase 2 Extension after study day 84 (D84).

FIG. 6A, and FIG. 6B provide data for the extension study generally depicted in FIG. 5 and described in Example 3. FIG. 6A shows the absolute glucose reading measurements obtained from the seven (7) control Group 1 mice remaining at study day 84 (D84). It is noted that of these remaining seven (7) mice, four (4) died on or before study day 112 (D112) and only three (3) remained alive as of study day 301 (D301). Thus, FIG. 6A shows that 70% of control mice became diabetic before week 14. FIG. 6B shows the absolute glucose reading measurements obtained from the seven (7) high dose ADI-100 Group 4 mice remaining at study day 84 (D84). It is noted that of these remaining seven (7) high dose ADI-100 group mice, one (1) died (subject number 247) on or around study day 196 (D196) and the remaining six (6) remained alive as of study day 301 (D301). It is also noted that two (2) mice (subject numbers 214 and 247) showed evidence of lost tolerance in the form of elevated absolute glucose reading measurements. Of these two mice, one was successfully treated and recovered (subject number 214), wherein the resumed treatment resulted in lowered or normalized absolute glucose reading measurements.

FIG. 7A, FIG. 7B, and FIG. 7C show results of a study run in the Hepa1-6 liver model using C57BL/6 mice to test the anti-tumor efficacy of vehicle alone, anti-PD-1 alone, and anti-PD-1 in conjunction with the high dose ADI-100 test article high dose ADI-100 (2 μg/μL in a ratio of BAX/msGAD of 1:2 (BAX 34 μg+msGAD 66 μg)). Anti-PD-1 is reported to be highly efficacious in the Hepa1-6 mouse tumor model. Thirty (30) mice were enrolled in the study. All animals were randomly allocated to the three (3) different study groups of 10 mice per group. FIG. 7A shows tumor volume measurements (mm³) over time (study days) with a mean absolute tumor volume±standard error of measurement (SEM). FIG. 7B shows body weight measurements (g) over time (study days) as a mean absolute body weight±SEM. FIG. 7C shows percent change in body weight (% change) over time (study days) as a mean percent change in body weight±SEM.

### DETAILED DESCRIPTION

### Overview

Using checkpoint inhibitors to treat cancer poses the risk of a flare up of preexistent autoimmune disease and the development of new autoimmune manifestations in patients with preexisting autoimmunity even if present as a biological only manifestation (as for example positive auto antibodies) or patients with no known autoimmunity disease.

To combat actual or potential autoimmune disease flare up and new autoimmune manifestations, antigen-specific treatment using an antigen-specific nucleic acid-based apoptotic DNA immunotherapy technology is disclosed herein. It is contemplated that administration of antigen-specific treatment using this nucleic acid-based apoptotic DNA immunotherapy technology may result in a targeted upregulation of regulatory T cells thereby addressing specific aspects of unhelpful autoimmune response caused by immune checkpoint drug therapy without impairing the tumor killing activity of other effector T cells. It is contemplated that patients or subjects receiving treatment may be tested before, during, or after treatment to assess actual or potential autoimmune disease flare up and new autoimmune manifestations. Such testing may facilitate, among other things, optimal patient selection, optimal dose selection, and/or optimal dosing regime selection.

### Antigen-Specific Treatment of Potential or Actual ICI Adverse Effects

Immunotherapy using ICIs has opened the door for a new approach to treat certain types of cancers by enabling effector T cells to see and destroy tumor cells that would otherwise be undetected. But use of checkpoint inhibitors has a broad effect such that removal of the “brakes” from these T cells is not tumor specific, and it is well known that immunological adverse effects may result. For example, a non-specific increase in effector T cell activity can result in autoimmunity in certain individuals who receive these new treatments. Antigen-specific treatment of these potential or actual ICI adverse effects using a nucleic acid-based apoptotic DNA immunotherapeutic technology (ADI™) a, or ADI-100 as described herein, may result in a targeted upregulation of regulatory T cells thereby addressing specific aspects of unhelpful autoimmune response without impairing the tumor killing activity of the effector T cells.

Described herein are new compositions and methods for preventing, mitigating, reducing, dampening, recovering from, reversing, or eliminating untoward antigen-specific or autoimmunity-inducing side effects resulting from use of ICIs. The presently described compositions and methods extrapolate from and build upon previously described compositions and methods for treating or reversing hyperglycemia and suppressing diabetes onset in a patient at risk of developing T1DM by administering a nucleic acid-based technology antigen-specific apoptotic DNA immunotherapeutic vector system comprising (a) a first expression cassette encoding BCL2 associated X apoptosis regulator (BAX); and (b) a hypermethylated second expression cassette encoding a secreted form of glutamic acid decarboxylase 65 (e.g., sGAD55).

It is noted that varying degrees of methylation may be accomplished by use of, for example, bacterial or enzymatic enzymatic methylation. Enzymatic methylation may be accomplished using the methods and techniques disclosed, for example, in published international patent application WO 2023034727A1, titled Enzymatically methylated dna and methods of production and therapeutic use.

When this apoptotic DNA immunotherapy is administered to the patient, it may induce a tolerogenic response, which results in an increase in tolerogenic dendritic cell populations in draining lymph nodes as well as an increase in numbers of GAD-specific regulatory T cells. And this immunotherapy, and ADI-100 specifically, has been shown to be efficacious in reversing hyperglycemia and suppressing onset of type 1 diabetes in non-obese diabetic (NOD) mice. Alleva D G, Rezace M, Yip L, Ren G, Rosenberg J, Concepcion W, Escher A, Shabahang S, Thakor A S. Reversal of Hyperglycemia and Suppression of Type 1 Diabetes in the NOD Mouse with Apoptotic DNA Immunotherapy™ (ADi™), ADi-100. Biomedicines. 2020 Mar. 4; 8 (3): 53. doi: 10.3390/biomedicines8030053.

Without intervention, NOD/ShiLtJ mice are characterized by hyperglycemia and insulitis, a leukocytic infiltration of the pancreatic islets and typically become diabetic by 30 weeks of age (86% of females; 48% of males), with median age of onset in females at 18 weeks of age. Diabetes in NOD mice is characterized by hyperglycemia and insulitis, a leukocytic infiltration of the pancreatic islets. Marked decreases in pancreatic insulin content occur in females at about 12 weeks of age and several weeks later in males. Immune phenotypes in the NOD background consist of defects in antigen presentation, T lymphocyte repertoire, NK cell function, macrophage cytokine production, wound healing, and C5 complement. These defects make the NOD background a common choice for immunodeficient mouse strains. See The Jackson Laboratory website, available at: 001976-NOD Strain Details (jax.org).

Acceleration of a diabetic state in the NOD/ShiLtJ mouse model can be achieved by inhibition of PD1-PDL1 signaling in NOD mice to accelerate onset of type 1 diabetes, implicating this pathway in suppressing the emergence of pancreatic beta cell reactive T-cells. Kochupurakkal N M, Kruger A J, Tripathi S, Zhu B, Adams L T, Rainbow D B, Rossini A, Greiner D L, Sayegh M H, Wicker L S, Guleria I. Blockade of the programmed death-1 (PD1) pathway undermines potent genetic protection from type 1 diabetes. PLOS One. 2014 Feb. 28; 9 (2): e89561. doi: 10.1371/journal.pone.0089561. PMID: 24586872; PMCID: PMC3938467. It has been determined that, in the NOD mouse model, anti-PD-L1 but not anti-CTLA-4 induced diabetes rapidly. Perdigoto A L, Deng S, Du K C, Kuchroo M, Burkhardt D B, Tong A, Israel G, Robert M E, Weisberg S P, Kirkiles-Smith N, Stamatouli A M, Kluger H M, Quandt Z, Young A, Yang M L, Mamula M J, Pober J S, Anderson M S, Krishnaswamy S, Herold K C. Immune cells and their inflammatory mediators modify β cells and cause checkpoint inhibitor-induced diabetes. JCI Insight 2022; 7 (17): e156330 https://doi.org/10.1172/jci.insight. 156330. Further it has been found that in these accelerated NOD mouse models, ICIs targeting the PD-1/PD-L1 pathway resulted in transcriptional changes in B cells and immune infiltrates that may lead to the development of diabetes, and that immune cells and their inflammatory mediators modify β cells and cause ICI-induced diabetes. Id. It has been found that treatment with anti-IFN-γ and anti-TNF-α prevented TIDM in anti-PD-L1-treated NOD mice, and suggested that inhibition of inflammatory cytokines can prevent TIDM as a strategy for clinical application to prevent this complication. Id.

For the first time, as described here, the inventors demonstrate that unwanted immunological side effects resulting from use of ICIs can be effectively blocked by use of an antigen-specific apoptotic DNA immunotherapy. And use of an antigen-specific apoptotic DNA immunotherapy can be used to preventing, mitigating, reducing, dampening, recovering from, reversing, or eliminating untoward antigen-specific side effects resulting from use of ICIs. Specifically, it is shown that development of T1DM is blocked in an accelerated polygenic NOD/ShiLtJ mouse model for autoimmune type 1 diabetes involving both an anti-PD1 antibody and antigen-specific apoptotic DNA immunotherapy.

Also, for the first time, as described here, the inventors demonstrate that use of an antigen-specific apoptotic DNA immunotherapy does not block the effectiveness of ICIs. That is, anti-tumor efficacy of an antigen-specific apoptotic DNA immunotherapy is tested in the Hepa1-6 model in C57BL/6 mice, reported to be highly efficacious in the Hepa1-6 mouse tumor model. Specifically, and as described further herein, the inventors test the anti-tumor efficacy of vehicle alone, anti-PD-1 alone, and anti-PD-1 in conjunction with the high dose ADI-100 test article in the Hepa1-6 mouse tumor model. And it was found that both the anti-PD-1 alone group and the anti-PD-1+ADI-100 group had greater than eighty percent (>80%) tumor inhibition compared to the vehicle alone control group. Thus, ADI-100 in anti-PD-1+ADI-100 group did not negatively interfere with the efficacy displayed by anti-PD-1 compared to anti-PD-1 alone group. It is further noted that the ADI-100 did not show any signs of toxicity.

Through experimentation it has been shown that ADI-100, comprised of two plasmids, one encoding BAX and another hypermethylated plasmid encoding a secreted form of GAD (sGAD55) formulated in a 1:2 ratio, addresses anti-PD1-Ab-accelerated autoimmune diabetes in a Non-Obese Diabetic NOD-ShiLtj mouse model of type 1 diabetes. These findings indicate that unwanted effects of checkpoint immune inhibitors may be mitigated, reduced, or eliminated by using an antigen-specific apoptotic DNA immunotherapeutic. It is believed that antigen-specific apoptotic DNA immunotherapy, here ADI-100, may result in a targeted upregulation of regulatory T cells thereby addressing specific aspects of unhelpful autoimmune response without impairing the tumor killing activity of the effector T cells.

As described and exemplified herein, ADI-100 is used to address anti-PD1-Ab-accelerated autoimmune diabetes in an animal model for type 1 diabetes and is demonstrated to suppress hyperglycemia and diabetes onset. The current findings build upon prior methods of using ADI-100 to treat type 1 diabetes. See Alleva D G, Rezaee M, Yip L, Ren G, Rosenberg J, Concepcion W, Escher A, Shabahang S, Thakor A S. Reversal of Hyperglycemia and Suppression of Type 1 Diabetes in the NOD Mouse with Apoptotic DNA Immunotherapy™ (ADi™), ADi-100. Biomedicines. 2020 Mar. 4; 8 (3): 53. doi: 10.3390/biomedicines8030053 and published U.S. patent application US 2024/0016905.

The ADI-100 vector system comprises or consists of (a) a first expression cassette encoding BCL2 associated X apoptosis regulator (BAX); and (b) a second hypermethylated expression cassette encoding a secreted glutamic acid decarboxylase 65 (e.g., sGAD55) which are administered to the patient to induce a tolerogenic response, which may include increasing tolerogenic dendritic cell populations in draining lymph nodes as well as increasing numbers of GAD-specific regulatory T cells. Id., and which may be in a pharmaceutically acceptable formulation.

In one aspect, a composition and method of preventing or reversing hyperglycemia in a patient at risk of developing type 1 diabetes is provided, the composition and method comprising administering a checkpoint inhibitor and a therapeutically effective amount of a vector system comprising (a) a first expression cassette comprising a polynucleotide encoding BAX; and (b) a hypermethylated second expression cassette comprising a polynucleotide encoding a secreted form of glutamic acid decarboxylase 65 (GAD65), which may be in a pharmaceutically acceptable formulation.

In another aspect, a composition and method of suppressing diabetes onset in a patient at risk of developing type 1 diabetes is provided, the composition and method comprising administering a checkpoint inhibitor and a therapeutically effective amount of a vector system comprising (a) a first expression cassette comprising a polynucleotide encoding BAX; and (b) a hypermethylated second expression cassette comprising a polynucleotide encoding a secreted form of glutamic acid decarboxylase 65 (GAD65).

In yet another aspect, a composition and method of increasing numbers of tolerogenic dendritic cells and GAD-specific regulatory T cells in a patient at risk of developing type 1 diabetes is provided, the composition and method comprising administering a checkpoint inhibitor and an effective amount of a vector system comprising a first expression cassette comprising a polynucleotide encoding BCL2 associated X apoptosis regulator (BAX) and a second expression cassette comprising a hypermethylated polynucleotide encoding a secreted form of glutamic acid decarboxylase 65 (e.g., sGAD55). In any of the aforementioned embodiments, the first expression cassette may further comprise a promoter operably linked to the polynucleotide encoding the BAX and the second expression cassette may further comprise a promoter operably linked to the polynucleotide encoding the secreted form of GAD65. In certain embodiments, the first expression cassette comprises a CMV promoter or an SV-40 promoter operably linked to the polynucleotide encoding the BAX. In certain embodiments, the second expression cassette comprises an SV-40 promoter operably linked to the polynucleotide encoding the secreted form of GAD65.

In any of the aforementioned embodiments, the secreted form of GAD65 may be encoded by msGAD55.

In any of the aforementioned embodiments, the vector system may comprise (a) a first vector comprising the first expression cassette expressing BAX; and (b) a hypermethylated second vector comprising the second expression cassette expressing the secreted form of GAD65. In some embodiments, the first vector and the second vector are administered at a ratio ranging from 1:1 to 1:8, including any ratio within this range such as 1:1, 1:2, 1:3, 1:4, 1:5, 1:6, 1:7, or 1:8. In some embodiments, the first vector and the second vector are administered at a ratio of 1:2.

In any of the aforementioned embodiments, the patient may have mild hyperglycemia, moderate hyperglycemia, or severe hyperglycemia. In certain embodiments, the patient has severe hyperglycemia and the first vector and the second vector are administered at a ratio of 1:2.

In any of the aforementioned embodiments, the patient may have an amount of insulin-producing pancreatic beta cells less than 50%, less than 60%, less than 70%, or less than 80% of a reference amount of beta cells for a non-diabetic subject. In some embodiments, the patient has lost 50% to 80% of the beta cells, including any amount within this range such as 50%, 55%, 60%, 65%, 70%, 75%, or 80% of the beta cells.

In any of the aforementioned embodiments, the patient may be human or non-human.

In another aspect, a composition for and method of increasing numbers of tolerogenic dendritic cells and GAD-specific regulatory T cells in a patient at risk of developing type 1 diabetes is provided, the composition and method comprising administering a checkpoint inhibitor and an effective amount of a vector system comprising a first expression cassette comprising a polynucleotide encoding BCL2 associated X apoptosis regulator (BAX) and a second expression cassette comprising a hypermethylated polynucleotide encoding a secreted form of glutamic acid decarboxylase 65 (e.g., sGAD55).

ICIs include, but are not limited to, those that target CTLA-4 (Ipilimumab); PD-1 (Cimiplimab; Nivolumab; Pembrolizumab); PD-L1 (Atezolizumab; Avelumab; Durvalumab); LAG-3 also known as CD223 (LAG525-IMP701, REGN3767-R3767), BI 754,091, tebotelimab-MGD013, eftilagimod alpha-IMP321, FS118; TIM-3 (MBG453, Sym023, TSR-022); by-h3, b7-h4 (MGC018, FPA150); A2aR (EOS100850, AB928); CD73 (CPI-006); NKG2A (Monalizumab); PVRIG/PVRL2 (COM701); CEACAMI (CM24); CEACAM 5/6 (NEO-201); FAK (Defactinib); CCL2/CCR2 (PF-04136309); LIF (MSC-1); CD47/SIRPa (Hu5F9-G4 (5F9), ALX148, TTI-662, RRx-001); CSF-1 also known as M-CSF/CSF-1R (Lacnotuzumab-MCS110), LY3022855, SNDX-6352, emactuzumab-RG7155), pexidartinib-PLX3397); IL-1 and IL-1R3 also known as IL-1RAP (CAN04, Canakinumab-ACZ885); IL-8 (BMS-986253); SEMA4D (Pepinemab-VX15/2503); Ang-2 (Trebananib); CLEVER-1 (FP-1305); Axl (Enapotamab vedotin-EnaV); and Phosphatidylserine (Bavituximab).

Before the present compositions, methods, and kits are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order that is logically possible.

It must be noted that as used herein and in the appended claims, the singular forms “a”, “an”, and “the” include plural referents unless the context clearly dictates otherwise. Thus, for example, reference to “a vector” includes a plurality of such vectors and reference to “the cell” includes reference to one or more cells, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates that may need to be independently confirmed.

### Definitions

“Tolerogenic” means capable of suppressing or down-modulating an adaptive immunological response.

The term “tolerogenic dendritic cell” refers to a dendritic cell that has the ability to induce immunological tolerance. A tolerogenic dendritic cell has low ability to activate effector T cells but high ability to induce and activate regulatory T cells.

“Recombinant” as used herein to describe a nucleic acid molecule means a polynucleotide of genomic, cDNA, viral, semisynthetic, or synthetic origin that, by virtue of its origin or manipulation, is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

The term “transformation” refers to the insertion of an exogenous polynucleotide into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

“Recombinant host cells,” “host cells,” “cells”, “cell lines,” “cell cultures,” and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

A “coding sequence” or a sequence that “encodes” a selected polypeptide, is a nucleic acid molecule which is transcribed (in the case of DNA) and translated (in the case of mRNA) into a polypeptide in vivo when placed under the control of appropriate regulatory sequences (or “control elements”). The boundaries of the coding sequence can be determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

“Operably linked” refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Encoded by” refers to a nucleic acid sequence which codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence.

“Expression cassette” or “expression construct” refers to an assembly that is capable of directing the expression of the sequence(s) or gene(s) of interest. An expression cassette generally includes control elements, as described above, such as a promoter which is operably linked to (so as to direct transcription of) the sequence(s) or gene(s) of interest, and often includes a polyadenylation sequence as well. Within certain embodiments of the invention, the expression cassette described herein may be contained within a plasmid construct. In addition to the components of the expression cassette, the plasmid construct may also include, one or more selectable markers, a signal which allows the plasmid construct to exist as single stranded DNA (e.g., a M13 origin of replication), at least one multiple cloning site, and a “mammalian” origin of replication (e.g., a SV40 or adenovirus origin of replication).

“Purified polynucleotide” refers to a polynucleotide of interest or fragment thereof that is essentially free, e.g., contains less than about 50%, preferably less than about 70%, and more preferably less than about at least 90%, of the protein with which the polynucleotide is naturally associated. Techniques for purifying polynucleotides of interest are well-known in the art and include, for example, disruption of the cell containing the polynucleotide with a chaotropic agent and separation of the polynucleotide(s) and proteins by ion-exchange chromatography, affinity chromatography and sedimentation according to density.

The term “transfection” is used to refer to the uptake of foreign DNA by a cell. A cell has been “transfected” when exogenous DNA has been introduced inside the cell membrane. A number of transfection techniques are generally known in the art. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous DNA moieties into suitable host cells. The term refers to both stable and transient uptake of the genetic material, and includes uptake of peptide- or antibody-linked DNAs.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells. Gene delivery expression vectors include, but are not limited to, vectors derived from bacterial plasmid vectors, viral vectors, non-viral vectors, alphaviruses, pox viruses and vaccinia viruses.

A polynucleotide “derived from” a designated sequence refers to a polynucleotide sequence which comprises a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

A “reference level” or “reference value” of a biomarker means a level of the biomarker (e.g., blood glucose level or number of pancreatic beta islets) that is indicative of a particular disease state, phenotype, or predisposition to developing a particular disease state or phenotype, or lack thereof, as well as combinations of disease states, phenotypes, or predisposition to developing a particular disease state or phenotype, or lack thereof. A “positive” reference level of a biomarker means a level that is indicative of a particular disease state or phenotype. A “negative” reference level of a biomarker means a level that is indicative of a lack of a particular disease state or phenotype. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Appropriate positive and negative reference levels of biomarkers for a particular disease state, phenotype, or lack thereof may be determined by measuring levels of desired biomarkers in one or more appropriate subjects, and such reference levels may be tailored to specific populations of subjects (e.g., a reference level may be age-matched or gender-matched so that comparisons may be made between biomarker levels in samples from subjects of a certain age or gender and reference levels for a particular disease state, phenotype, or lack thereof in a certain age or gender group). Such reference levels may also be tailored to specific techniques that are used to measure levels of biomarkers in samples (e.g., fluorescence-activated cell sorting (FACS), immunoassays (e.g., ELISA), mass spectrometry (e.g., LC-MS, GC-MS), tandem mass spectrometry, NMR, biochemical or enzymatic assays, PCR, microarray analysis, etc.), where the levels of biomarkers may differ based on the specific technique that is used.

The terms “quantity”, “amount”, and “level” are used interchangeably herein and may refer to an absolute quantification of a molecule, cell (e.g., pancreatic islets), or an analyte in a sample, or to a relative quantification of a molecule or analyte in a sample, i.e., relative to another value such as relative to a reference value as taught herein, or to a range of values for the biomarker. These values or ranges can be obtained from a single patient or from a group of patients.

“Diagnosis” as used herein generally includes determination as to whether a subject is likely affected by a given disease, disorder or dysfunction. The skilled artisan often makes a diagnosis on the basis of one or more diagnostic indicators, i.e., a biomarker, the presence, absence, or amount of which is indicative of the presence or absence of the disease, disorder or dysfunction. “Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression or reversal of the disease and/or symptom(s). Those in need of treatment include those already afflicted (e.g., those with hyperglycemia or pre-diabetic) as well as those in which prevention is desired (e.g., those with increased susceptibility to diabetes, those having a genetic predisposition to developing diabetes, etc.). The terms “treatment”, “treating”, “treat” and the like may encompass suppression of diabetes onset.

The term “suppressing diabetes onset” is a type of treatment used herein to generally refer to preventing or delaying the onset of diabetes. Delaying the onset of diabetes includes delay for one or more days, one or more weeks, one or more months, or longer. Preventing the onset of diabetes includes preventing the onset of diabetes over a specific time period or preventing the onset of diabetes over an indefinite period of time. The onset of diabetes may be identified by any appropriate measurement, such as measurement of blood glucose levels, measurement of insulin production, etc.

Hyperglycemia, as used herein, refers to the condition of having excess glucose in the bloodstream. Hyperglycemia is also referred to as prediabetes or stage 2 disglycemia. Hyperglycemia may be characterized as mild, moderate, or severe, based on blood sugar levels. For people without diabetes, a healthy fasting blood sugar level is about 70 to 100 milligrams per deciliter of blood (mg/dL). Hyperglycemia is diagnosed when fasting blood sugar levels are between about 100 mg/dL and 125 mg/dL. Fasting blood sugar greater than 126 mg/dL indicates the development of clinical diabetes. In the NOD mouse model, mild hyperglycemia refers to hyperglycemia wherein fasting blood glucose levels or morning blood glucose levels are about 140 mg/dL and severe hyperglycemia refers to hyperglycemia wherein fasting blood glucose levels or morning blood glucose levels are about 180 mg/dL or higher. An individual with severe hyperglycemia may also be referred to as “highly hyperglycemic.” Moderate hyperglycemia refers to hyperglycemia wherein fasting or morning blood glucose levels are in the range between mild and severe hyperglycemia, for example, between about 140 mg/dL and about 180 mg/dL in the NOD mouse model.

A therapeutic treatment is one in which the subject is afflicted prior to administration and a prophylactic treatment is one in which the subject is not afflicted prior to administration. In some embodiments, the subject has an increased likelihood of becoming inflicted or is suspected of being afflicted prior to treatment. In some embodiments, the subject is suspected of having an increased likelihood of becoming afflicted. Methods for administration of therapeutic treatments are well known in the art, and include oral, topical, transdermal or intradermal, inhalation, parenteral, sublingual, buccal, rectal, vaginal, and intranasal. The term “parenteral”, as used herein, includes subcutaneous injections (including, for example, transdermal or intradermal injections), intravenous, intramuscular, intrasternal injection or infusion techniques. In certain embodiments, administering comprises administering by a route that is selected from intradermal and mucosal.

The term “about,” particularly in reference to a given quantity, is meant to encompass deviations of plus or minus five percent.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. In some embodiments, the mammal is human.

A “therapeutically effective dose” or “therapeutic dose” is an amount sufficient to effect desired clinical results (i.e., achieve therapeutic efficacy). A therapeutically effective dose can be administered in one or more administrations.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer. Both full-length proteins and fragments thereof are encompassed by the definition. The terms also include postexpression modifications of the polypeptide, for example, phosphorylation, glycosylation, acetylation, hydroxylation, oxidation, and the like.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), and any other type of polynucleotide which is an N- or C-glycoside of a purine or pyrimidine base. There is no intended distinction in length between the terms “polynucleotide,” “oligonucleotide,” “nucleic acid” and “nucleic acid molecule,” and these terms are used interchangeably.

By “isolated” is meant, when referring to a protein, polypeptide, or peptide, that the indicated molecule is separate and discrete from the whole organism with which the molecule is found in nature or is present in the substantial absence of other biological macromolecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

### Antigen-Specific Apoptotic DNA Immunotherapy.

### Described Herein is a Unique and Potent Immunotherapy.

The immunotherapy may comprise two DNA plasmids, one expressing the intracellular apoptosis-inducing signaling molecule, BAX, and the other expressing the islet autoantigen, secreted glutamic acid decarboxylase 65 (sGAD55), with specific embodiments referred to and exemplified herein as “ADI-100”. Li, A. F.; Escher, A. DNA vaccines for transplantation; Li, A. F.; Escher, A. DNA vaccines for transplantation. Expert Opin. Biol. Ther 2010, 10, 903-915, doi: 10.1517/14712591003796546; Li, A.; Ojogho, O.; Franco, E.; Baron, P.; Iwaki, Y.; Escher, A. Pro-apoptotic DNA vaccination ameliorates new onset of autoimmune diabetes in NOD mice and induces foxp3+ regulatory T cells in vitro. Vaccine 2006, 24, 5036-5046, doi: 10.1016/j. vaccine.2006.03.041 (“Escher 2006”); Li, A.; Chen, J.; Hattori, M.; Franco, E.; Zuppan, C.; Ojogho, O.; Iwaki, Y.; Escher, A. A therapeutic DNA vaccination strategy for autoimmunity and transplantation. Vaccine 2010, 28, 1897-1904, doi: 10.1016/j.vaccine.2009.10.090 (“Escher 2010 Vaccination Strategy”).

It was previously shown that the efficacy of ADI-100 in the non-obese diabetic (NOD) mouse model of TID is significantly increased if the sGAD55 plasmid is hyper-methylated, which may reduce inflammation caused by unmethylated CpG motifs that are ligands for the Toll-like receptor 9 expressed on some APCs. See Escher 2010 Vaccination Strategy. ADI-100 treatment also increases sGAD-specific Treg levels in draining lymph nodes of NOD mice along with total CDIIc⁺DCs; though it is not known whether these DCs have a tolerogenic phenotype. Escher 2006; Escher 2010 Vaccination Strategy; and Li, A. F.; Hough, J.; Henderson, D.; Escher, A. Co-delivery of pro-apoptotic BAX with a DNA vaccine recruits dendritic cells and promotes efficacy of autoimmune diabetes prevention in mice. Vaccine 2004, 22, 1751-1763, doi: 10.1016/j.vaccine.2003.10.049. And it was found that ADi-100 treatment increases tolerogenic DCs (tol-DCs), and increasing the apoptosis-inducing BAX content enhances the efficacy in reversing hyperglycemia when administered to NOD mice during late hyperglycemia, a prediabetes stage that has relevance to the corresponding clinical diagnosis stage in human TID. Alleva D G, Rezaee M, Yip L, Ren G, Rosenberg J, Concepcion W, Escher A, Shabahang S, Thakor AS. Reversal of Hyperglycemia and Suppression of Type 1 Diabetes in the NOD Mouse with Apoptotic DNA Immunotherapy™ (ADi™), ADI-100. Biomedicines. 2020 Mar. 4; 8 (3): 53. doi: 10.3390/biomedicines8030053; See e.g., International Patent Cooperation Treaty patent application PCT/US2021/020711, titled “Methods of treating hyperglycemia and suppressing onset of type 1 diabetes” and published as WO2021178565.

### ADi-100: Plasmid DNA Construct.

The two DNA plasmids that comprise the ADI-100 formulation previously described and exemplified herein are pND2-BAX containing a bax cDNA sequence under transcriptional control of the CMV promoter and pSG5-GAD55 containing a cDNA construct encoding a secreted form of human GAD65 (sGAD55) under transcriptional control of the SV-40 promoter in the pSG5 vector (Stratagene, San Diego, CA, USA). Escher 2010 Vaccination Strategy. The pSG5-GAD plasmid was hyper-methylated at CpG motifs (msGAD55) in Escherichia coli strain, ER1821, via the activity of Sssl methylase (New England BioLabs, Ipswich, MA, USA). This method has been shown to result in 85%-100% methylation of CpG motifs in a plasmid (see Jimenez-Useche et al., Biophys J. 107 (7) 1629-1636). It is contemplated, however, that enzymatic methylation may also be used to achieve various levels of methylation of CpG motifs in a plasmid. Plasmid DNA was dissolved in sterile saline immediately prior to intradermal (i.d.) injection. All plasmids containing the BAX sequence insert showed significant and substantial degrees of apoptosis of human HeLa cells (using 1 ug/mL DNA in cultures; data not shown), confirming the activity of the BAX-induced apoptosis tolerance delivery system of ADI-100.

Through experimentation it has been shown that ADI-100, comprised of two plasmids, one encoding BAX and another hypermethylated plasmid encoding a secreted form of GAD (sGAD55) formulated in a 1:2 ratio, addresses anti-PD1-Ab-accelerated autoimmune diabetes in a Non-Obese Diabetic NOD-ShiLtj mouse model of type 1 diabetes.

### Immune Checkpoint Inhibition.

Immune checkpoints are inhibitory receptors that convey negative signals to immune cells, preventing autoimmunity. The importance of immune checkpoints in supporting tolerance and preventing autoimmunity development is best observed in knockout mice models. For instance, the lack of CTLA-4, PD-1, BTLA (B- and T-lymphocyte attenuator), TIGIT (T-cell immunoreceptor with immunoglobulin and ITIM domain), and VISTA (V-domain Ig suppressor of T-cell activation) was shown to cause massive lymphoproliferation, an onset of autoimmune diseases, or fatal multiorgan tissue destruction (notably CTLA-4 deficiency). In humans, several polymorphisms of immune checkpoint genes were identified and reported to be associated with susceptibility to autoimmune diseases. See e.g., Yu L, Shao M, Zhou T, Xie H, Wang F, Kong J, et al. Association of CTLA-4 (+49 A/G) Polymorphism With Susceptibility to Autoimmune Diseases: A Meta-Analysis With Trial Sequential Analysis. Int Immunopharmacol (2021) 96:107617. doi: 10.1016/j.intimp.2021.107617.

Programmed cell death receptor 1 (PD-1) is an immune checkpoint significant for self-tolerance and the cessation of the immune response that became a target of cancer immunotherapy. Upon engagement by its ligand (PD-L1, Programmed cell death ligand 1), PD-1 acts as a brake to the immune system that induces the apoptosis of activated T cells. Francisco L M, Sage P T, Sharpe A H. The PD-1 Pathway in Tolerance and Autoimmunity. Immunol Rev (2010) 236:219-42. doi: 10.1111/j.1600-065X.2010.00923.x. PD-L1 expression can be detected in pancreatic islets, vascular endothelial cells, and placenta where it is responsible for tissue protection from autoimmune responses. Keir M E, Liang S C, Guleria I, Latchman Y E, Qipo A, Albacker L A, et al. Tissue Expression of PD-L1 Mediates Peripheral T Cell Tolerance. J Exp Med (2006) 203:883-95. doi: 10.1084/jem.20051776. For example, in TID (type 1 diabetes), PD-L1 was observed to be upregulated in insulin-producing beta cells under an autoimmune attack and correlated with the intensity of CD8⁺ T-cell infiltration in the pancreas. Colli M L, Hill J L E, MarroquíL, Chaffey J, Dos Santos R S, Leete P, et al. PDL1 is Expressed in the Islets of People With Type 1 Diabetes and is Up-Regulated by Interferons-a and-g via IRF1 Induction. EBioMedicine (2018) 36:367-75. doi: 10.1016/j.ebiom.2018.09.040; Osum K C, Burrack A L, Martinov T, Sahli N L, Mitchell J S, Tucker C G, et al. Interferon-Gamma Drives Programmed Death-Ligand 1 Expression on Islet b Cells to Limit T Cell Function During Autoimmune Diabetes. Sci Rep (2018) 8:8295. doi: 10.1038/s41598-018-26471-9. In addition, PD-1/PD-L1 interaction was reported to be involved in the generation of inducible Tregs (iTregs). Francisco et al. showed that PD-L1-negative APCs (antigen-presenting cells) had an impaired ability to generate Tregs, either in vitro or in vivo. Francisco L M, Salinas V H, Brown K E, Vanguri V K, Freeman G J, Kuchroo V K, et al. PD-L1 Regulates the Development, Maintenance, and Function of Induced Regulatory T Cells. J Exp Med (2009) 206:3015-29. doi: 10.1084/jem.20090847. The failure of APCs isolated from systemic lupus erythematosus (SLE) patients to upregulate PD-L1 expression validates these findings in humans. Mozaffarian N, Wiedeman A E, Stevens A M. Active Systemic Lupus Erythematosus is Associated With Failure of Antigen-Presenting Cells to Express Programmed Death Ligand-1. Rheumatology (2008) 47:1335-41. doi: 10.1093/rheumatology/ken256.

The blockade of PD-1 or PD-L1 in experimental models of autoimmunity led to disease onset and exacerbation, indicating the essential role of these immune checkpoints in tolerance and, specifically, in Treg maintenance. Pauken K E, Jenkins M K, Azuma M, Fife B T. PD-1, But Not PD-L1, Expressed by Islet-Reactive CD4+ T Cells Suppresses Infiltration of the Pancreas During Type 1 Diabetes. Diabetes (2013) 62:2859-69. doi: 10.2337/db12-1475; 9. Ke Y, Sun D, Jiang G, Kaplan H J, Shao H. PD-L1 Hi Retinal Pigment Epithelium (RPE) Cells Elicited by Inflammatory Cytokines Induce Regulatory Activity in Uveitogenic T Cells. J Leukoc Biol (2010) 88:1241-9. doi: 10.1189/jlb.0610332. Recent reports on autoimmune-related adverse events in oncologic patients treated with PD-1/PD-L1 axis blockers support these findings. Zhao Z, Wang X, Bao X, Ning J, Shang M, Zhang D. Autoimmune Polyendocrine Syndrome Induced by Immune Checkpoint Inhibitors: A Systematic Review. Cancer Immunol Immunother (2021) 70:1527-40. doi: 10.1007/s00262-020-02699-1; Schneider S, Potthast S, Komminoth P, Schwegler G, Böhm S. PD-1 Checkpoint Inhibitor Associated Autoimmune Encephalitis. Case Rep Oncol (2017) 10:473-8. doi: 10.1159/000477162.

In cancer, effector T cells, which are persistently exposed to antigen stimulation in TME, express PD-1 at high levels, in the long term, causing T-cell functional exhaustion. It results in the inability of T cells to eliminate tumor cells and facilitates cancer progression. Additionally, cancer cells actively exploit PD-L1 to evade the immune system and hijack the immunosurveillance mechanisms with PD-L1 expression. Moreover, the results presented by Chen et al. (2018) revealed that apart from cell surface expression, PD-L1 was present in extracellular vesicles (exosomes) produced by melanoma cells, suggesting its systematic immunosuppressive impact. Chen G, Huang A C, Zhang W, Zhang G, Wu M, Xu W, et al. Exosomal PD-L1 Contributes to Immunosuppression and is Associated With Anti-PD-1 Response. Nature (2018) 560:382-6. doi: 10.1038/s41586-018-0392-8. As a result, it leads to the transcriptomic changes and the exhaustion of CD4⁺ and CD8⁺ T cells that are unable to eliminate cancer cells effectively. In a vast number of cancers, lymphocyte infiltration is in positive correlation with PD-L1 expression, which is simply an adaptive mechanism of the tumor to escape an immune response. Even though tumor PD-L1 expression usually suggests poor prognosis, then higher levels of tumor PD-L1 expression correlate with a better efficiency of immunotherapy. Iwai Y, Ishida M, Tanaka Y, Okazaki T, Honjo T, Minato N. Involvement of PD-L1 on Tumor Cells in the Escape From Host Immune System and Tumor Immunotherapy by PD-L1 Blockade. Proc Natl Acad Sci USA (2002) 99:12293-7. doi: 10.1073/pnas. 192461099.

It is noted that in Example 1, Example 2, and Example 3, described herein, the accelerated NOD/ShiLtJ mouse model for autoimmune type 1 diabetes involving both an anti-PD1 antibody and antigen-specific apoptotic DNA immunotherapy included an initial dose of 500 μg of anti-PD1 antibody which may have further accelerated the onset of a diabetic state as opposed to a lower dose of, for example, 250 μg.

The present disclosure provides the following embodiments:
Embodiment 1: A therapeutic composition comprising an immune checkpoint inhibitor and an apoptotic DNA immunotherapy.
Embodiment 2: The therapeutic composition of Embodiment 1, wherein the immune checkpoint inhibitor targets at least one of PD-1 and CTLA-4.
Embodiment 3: The therapeutic composition of Embodiment 1 or Embodiment 2, wherein the apoptotic DNA immunotherapy comprises a polynucleotide encoding BCL2 associated X apoptosis regulator (BAX).
Embodiment 4: The therapeutic composition of any one of Embodiments 1-3, wherein the apoptotic DNA immunotherapy comprises a polynucleotide encoding BCL2 associated X apoptosis regulator (BAX) and a polynucleotide encoding a secreted form of glutamic acid decarboxylase 65 (GAD65) and, optionally, wherein said polynucleotide encoding a secreted form of glutamic acid decarboxylase 65 (GAD65) is provided as a hypermethylated expression cassette.
Embodiment 5: A method of treating one or more adverse effects resulting from administration of an immune checkpoint inhibitor to a subject in need thereof comprising administration of an apoptotic DNA immunotherapy.
Embodiment 6: The method of Embodiment 5, further comprising administering at least one of an anti-PD-1 immune checkpoint inhibitor and an anti-CTLA-4 immune checkpoint inhibitor.
Embodiment 7: The method of Embodiment 5 or Embodiment 6, further comprising administering an apoptotic DNA immunotherapy comprising a polynucleotide encoding BCL2 associated X apoptosis regulator (BAX).
Embodiment 8: The method of any one of Embodiments 5-7, further comprising administering an apoptotic DNA immunotherapy comprises a polynucleotide encoding BCL2 associated X apoptosis regulator (BAX) and a polynucleotide encoding a secreted form of glutamic acid decarboxylase 65 (GAD65) and, optionally, wherein said polynucleotide encoding a secreted form of glutamic acid decarboxylase 65 (GAD65) is provided as a hypermethylated expression cassette.
Embodiment 9: The method of any one of Embodiments 5-8, wherein development of type 1 diabetes mellitus is prevented.
Embodiment 10: The method of any one of Embodiments 5-8, wherein hyperglycemia associated with development of type 1 diabetes mellitus is prevented or reduced.
Embodiment 11: The method of any one of Embodiments 5-10 further comprising administering the apoptotic DNA immunotherapy at any one or more times before, during, or after administration of an immune checkpoint inhibitor.
Embodiment 12: The method of any one of Embodiments 5-11 further comprising stopping administration of the apoptotic DNA immunotherapy at any one or more times before, during, or after administration of an immune checkpoint inhibitor upon indication that antigen-specific immune tolerance has been achieved in a subject in need thereof.
Embodiment 13: The method of any one of Embodiments 5-11 wherein the effectiveness of immune checkpoint inhibitor is maintained.
Embodiment 14: A method of preventing adverse effects resulting from administration of an immune checkpoint inhibitor to a subject in need thereof comprising administration of an apoptotic DNA immunotherapy.
Embodiment 15: The method of Embodiment 14, further comprising administering at least one of an anti-PD-1 immune checkpoint inhibitor and an anti-CTLA-4 immune checkpoint inhibitor.
Embodiment 16: The method of Embodiment 14 or Embodiment 15, further comprising administering an apoptotic DNA immunotherapy comprising a polynucleotide encoding BCL2 associated X apoptosis regulator (BAX).
Embodiment 17: The method of any one of Embodiments 14-16, further comprising administering an apoptotic DNA immunotherapy comprises a polynucleotide encoding BCL2 associated X apoptosis regulator (BAX) and a polynucleotide encoding a secreted form of glutamic acid decarboxylase 65 (GAD65) and, optionally, wherein said polynucleotide encoding a secreted form of glutamic acid decarboxylase 65 (GAD65) is provided as a hypermethylated expression cassette.
Embodiment 18: The method of any one of Embodiments 14-17, wherein development of type 1 diabetes mellitus is prevented.
Embodiment 19: The method of any one of Embodiments 14-17, wherein hyperglycemia associated with development of type 1 diabetes mellitus is prevented or reduced.
Embodiment 20: The method of any one of Embodiments 14-19 further comprising administering the apoptotic DNA immunotherapy at any one or more times before, during, or after administration of an immune checkpoint inhibitor.
Embodiment 21: The method of any one of Embodiments 14-20 further comprising stopping administration of the apoptotic DNA immunotherapy at any one or more times before, during, or after administration of an immune checkpoint inhibitor upon indication that antigen-specific immune tolerance has been achieved in a subject in need thereof.
Embodiment 22: The method of any one of Embodiments 14-21 wherein the effectiveness of immune checkpoint inhibitor is maintained.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed.

### Example 1

The Example 1 provides interim data and results from an ongoing study, wherein the objectives were two-fold: The first goal was to establish an accelerated type 1 diabetes (TID) mouse model in female Non-Obese Diabetic NOD-ShiLtj mice where disease is accelerated via Anti-PD-1 antibody treatment. The NOD-ShiLtj or NOD mouse strain is a polygenic autoimmune TID model that develops overt hyperglycemia due to immune-mediated pancreatic islet loss at ~12 weeks age in females. In this study, the impact of Anti-PD-1 antibody treatment on time of disease onset and disease progression was evaluated. The second goal was to determine the efficacy of ADI-100 BAX+msGAD treatment at two dose levels on the following parameters: blood glucose concentration (glycemia), pancreas gross morphology and islet histopathology, and pancreatic islet insulin content by immunohistochemistry.

In this study, 8-week-old female NOD-ShiLtj mice (JAX Strain #001976, Jackson Laboratories, Bar Harbor, ME) received anti-PD-1 antibody (BioXCell; Catalog #BE0033-2) or IgG isotype as control (BioXCell; Catalog #BE0091) every 2 days starting Day 0 and ending Day 10. These interim study findings, include tolerization with the DNA plasmids that was performed on Days 0, 7, 14 and 21.

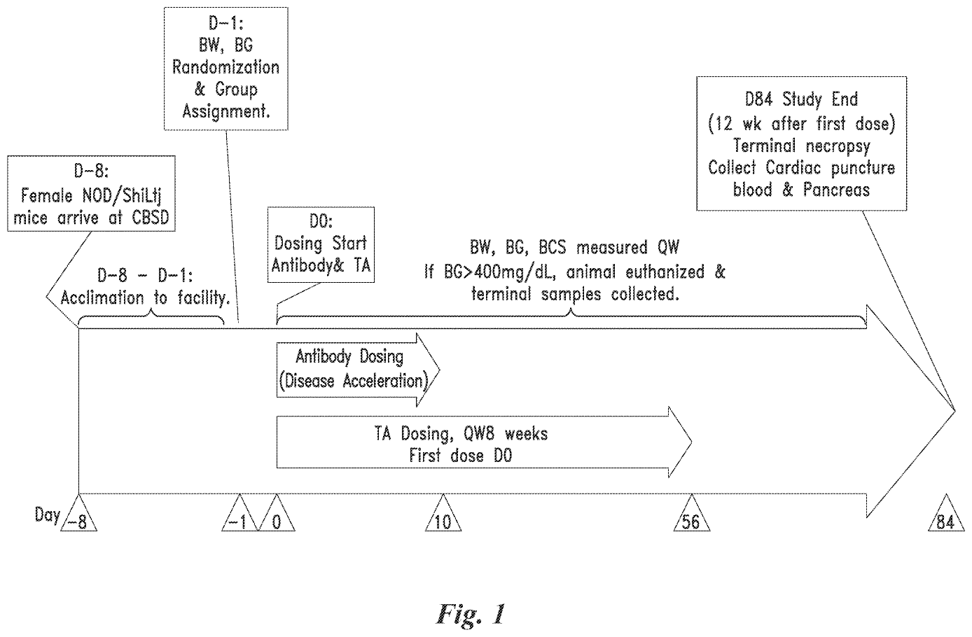

FIG. 1 and Table 1 below provide details of the study design and treatment groups for which these interim results were obtained.

FIG. 1 shows the study design and time course. 8-week-old mice were obtained and acclimated for 1 week. Following random assignment to one of the 4 groups, each animal received anti-PD1 antibody in an initial dose of 500 μg (day 0), followed by a dose of 250 μg (days 2, 4, 6, 8, 10) or IgG as negative control. As shown in FIG. 1, treatment using vehicle control or ADI-100 would be initiated on Day 0 and administered once a week (QW) for eight weeks. Blood glucose levels were monitored during treatment administration and for several weeks after cessation of treatment.

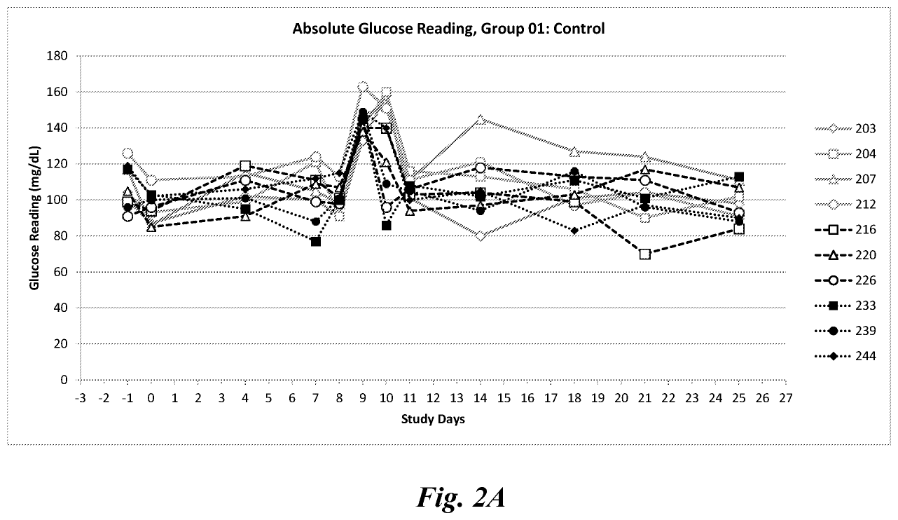

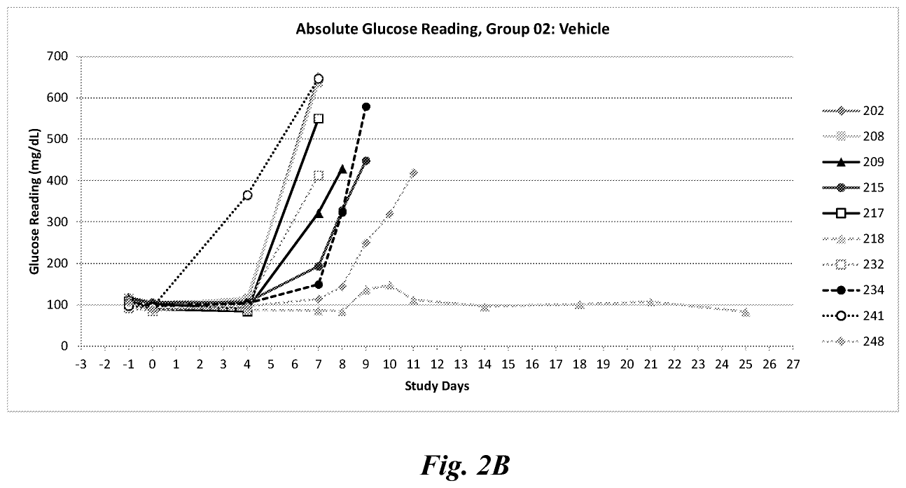

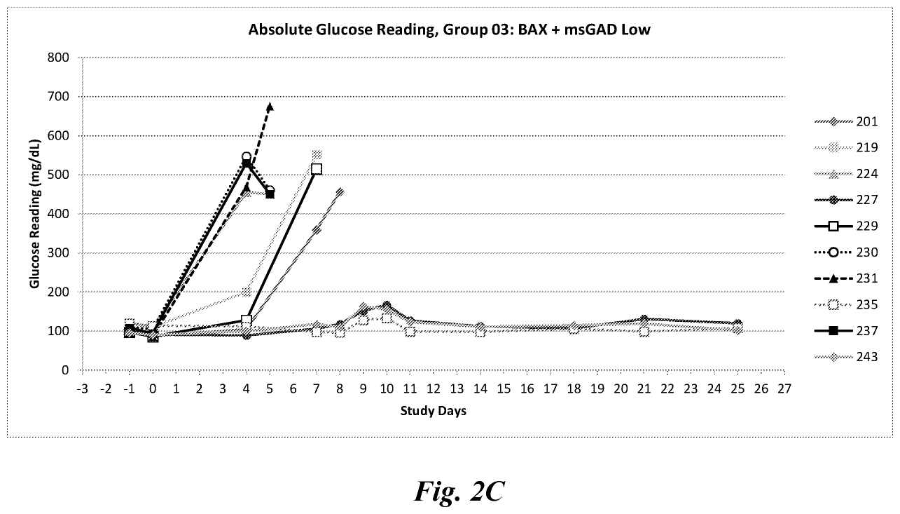

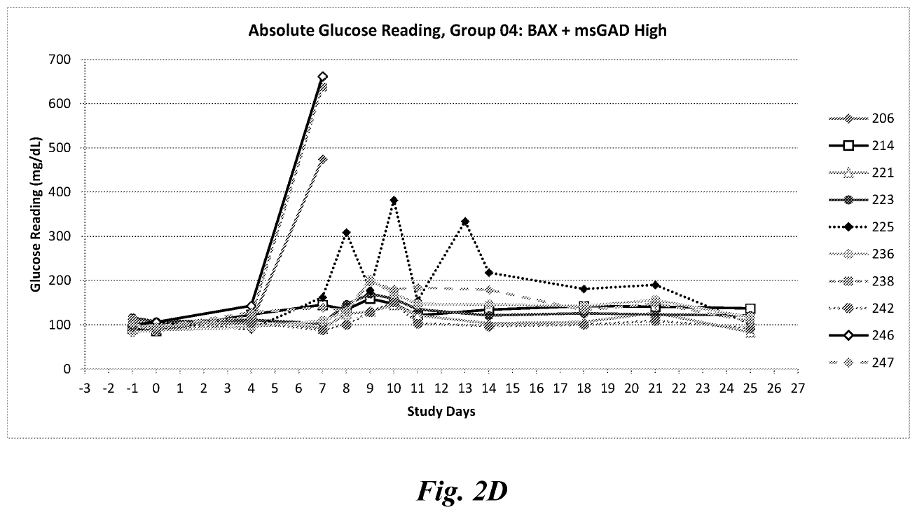

Table 1, below, shows the treatment groups and planned dosing regimen for the ongoing study from which these interim results for the first 25-days of the study were obtained. Forty female NOD mice were randomly assigned to 4 groups (N=10). Mice received 6 administrations of IgG (Group 1) or anti-PD1 antibody (Groups 2-4) starting with 500 μg on Day 0 and 250 μg on Days 2, 4, 6, 8 and 10. For these interim results, mice in Groups 2-4 also received the first four of the eight scheduled weekly administrations of tests articles on Days 0, 7, 14, and 21 as shown in the interim study data provided in FIG. 2A, FIG. 2B, FIG. 2C, and FIG. 2D. Group 2 animals received the vehicle control, Group 3 animals received 50 μL of ADI-100 at 1 μg/μL (low dose) in a ratio of BAX/msGAD of 1:2 and Group 4 animals received 50 μL of ADI-100 at 2 μg/μL (high dose) in a ratio of DNA BAX/msGAD of 1:2. ADI-100 1 μg/μL (low dose) in a ratio of BAX/msGAD of 1:2 (BAX 17 μg+msGAD 33 μg) was supplied by Aditxt, Inc., in a Tris-EDTA (TE) buffer. ADI-100 2 μg/μL (high dose) in a ratio of BAX/msGAD of 1:2 (BAX 34 μg+msGAD 66 μg) was supplied by Aditxt, Inc., in a Tris-EDTA (TE) buffer. Interperitoneal (IP) dose administration and intradermal (ID) dose administration are noted in Table 1, below.

| | IgG (Group 1) or | | | | | | |
|---|---|---|---|---|---|---|---|
| | Anti-PD1 antibody | | | | | | |
| | (Groups 2-4) treatment | | | | Dosing | | Dose |
| | Route, dose, | Test | | | Frequency & | Dose | Volume |
| Group | frequency, duration | Articles | N | Route | Duration | (mg/kg) | (μL) |
| Group 1 | IP | NA | 10 | NA | NA | NA | NA |
| IgG | Day 0: 500 μg; | | (each | | | | |
| isotype | | | Group) | | | | |
| control | | | | | | | |
| Group 2 | Day 2, 4, 6, 8, | Vehicle | | ID | Days 0, 7, 14, | 50 | 50 |
| Anti- | 10; 250 μg | | | | 21, 28 and | | |
| PD-1 | | | | | weekly for 8 | | |
| Group 3 | | Plasmid DNA | | ID | weeks or | 17 μg + | 50 |
| Anti- | | BAX + msGAD | | | until animals | 33 μg | |
| PD-1 | | | | | drop out | | |
| Low | | | | | | | |
| Dose | | | | | | | |
| Group 4 | | DNA | | ID | | 34 μg + | 50 |
| Anti- | | BAX + msGAD | | | | 66 μg | |
| PD-1 | | | | | | | |
| High | | | | | | | |
| Dose | | | | | | | |

Interim results of the experiment for the first 25-days of the study are shown in FIG. 2A, FIG. 2B, FIG. 2C, and FIG. 2D (absolute glucose readings). Mice in the control group (Group 1) showed relatively stable blood glucose levels for the 25-day observation period while most of the mice in Group 2 (9/10), which received anti-PD1 antibodies and vehicle showed overt hyperglycemia within 10 days demonstrating an earlier and more synchronized onset of disease. Most of the Group 3 mice (7/10) treated with low dose ADI-100 also showed hyperglycemia within the first 10 days. In contrast, seven out of ten (7/10) Group 4 mice treated with high dose ADI-100 showed normal glycemic levels during the observation period.

Any mouse with a BG measurement >400 mg/dL was euthanized according to study protocol. FIG. 2A shows that all control mice in Group 1 survived the first 25-days of the study. FIG. 2B shows that 9 of the 10 mice in Group 2 experienced a BG measurement >400 mg/dL within the first 11 days. FIG. 2C shows that 7 of the 10 mice in low dose test article Group 3 experienced a BG measurement >400 mg/dL within the first 8 days. FIG. 2D shows that 3 of the 10 mice in high dose test article (Group 4) experienced a BG measurement >400 mg/dL within the first 7 days (one week).

It is noted that, as shown in FIG. 2D, Group 4 (high dose) mouse subject 225 had multiple blood glucose (BG) readings of vacillating above and below 200 mg/dLon Study Days 7, 9, and 11 to greater than 250 mg/dL on Study Days 8, 10, and 13; however mouse subject 225 demonstrated normal glycemic levels following Study Day 14 through Study Day 25. These data support the effectiveness of ADI-100 in controlling the effect of anti-PD1 antibodies in accelerating autoimmune diabetes in NOD mice. These data also show that the effectiveness of ADI-100 is dose dependent.

### Example 2

This Example 2 provides further data obtained from the continuation of Example 1, described above.

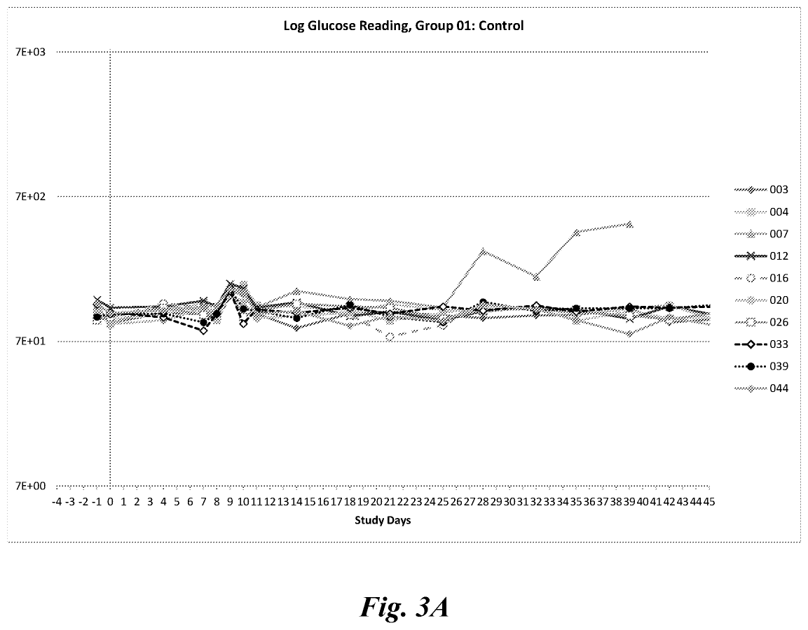

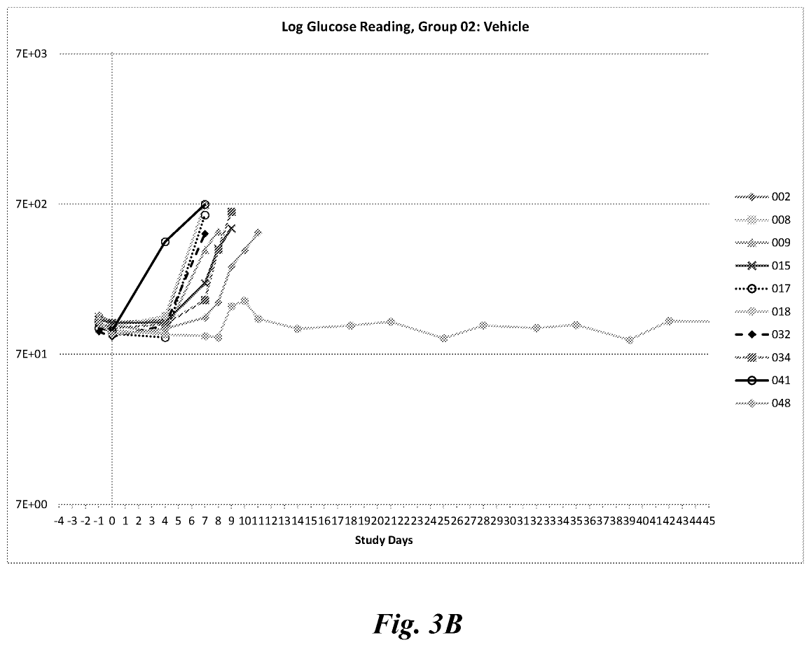

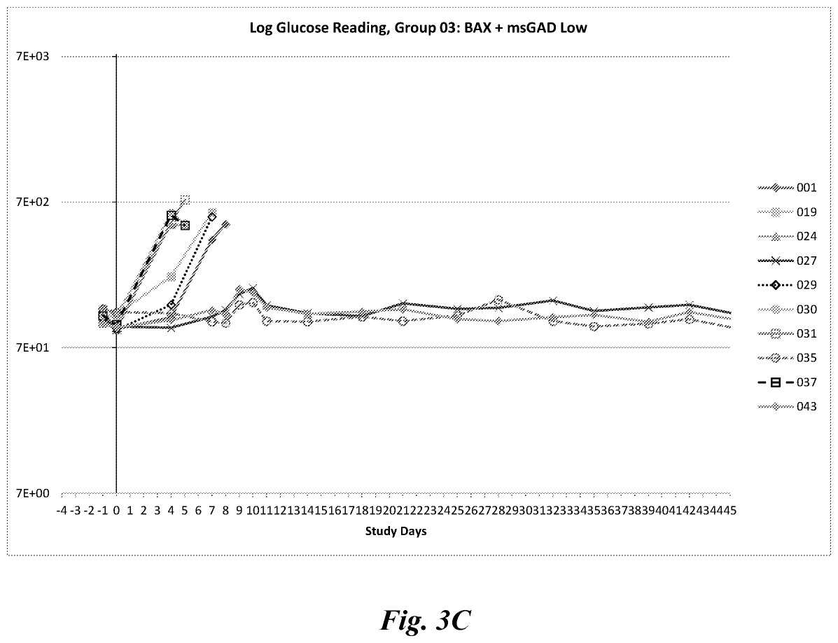

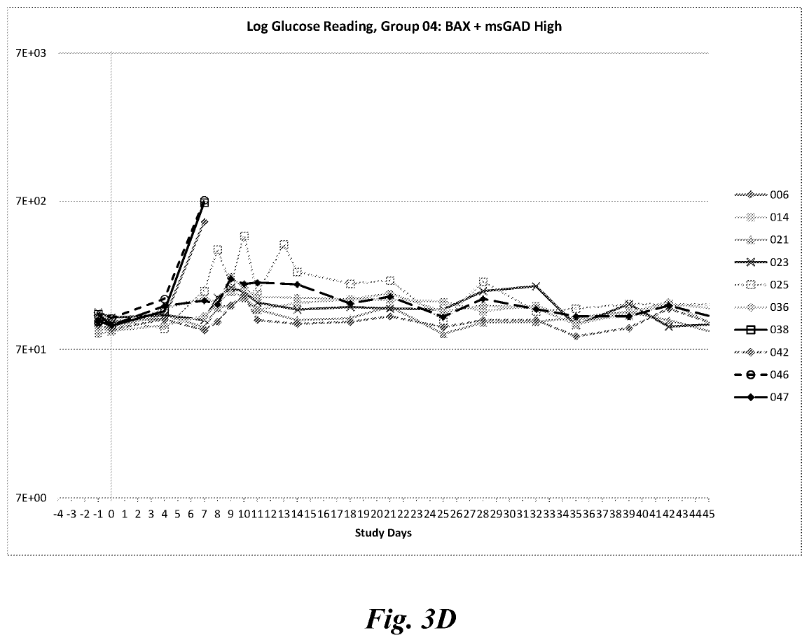

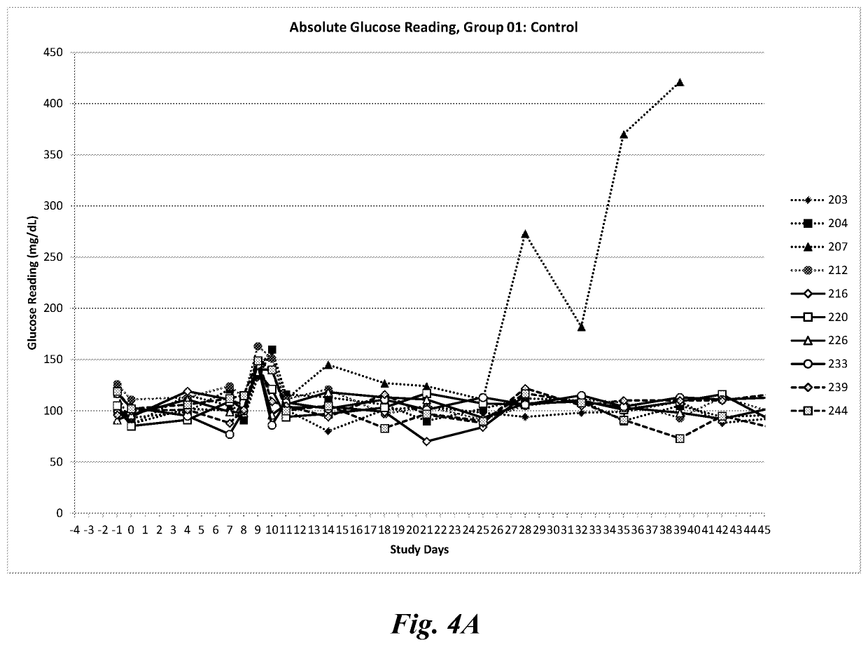

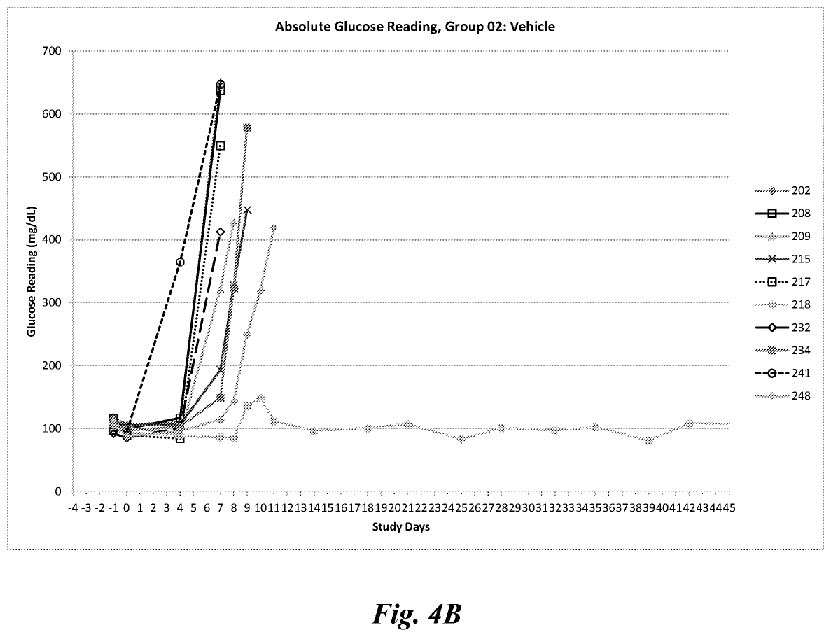

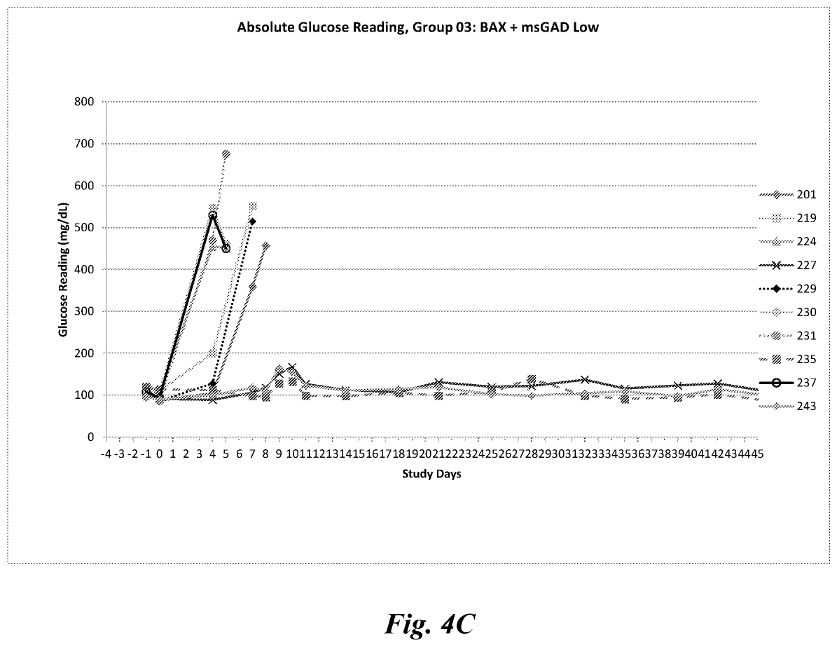

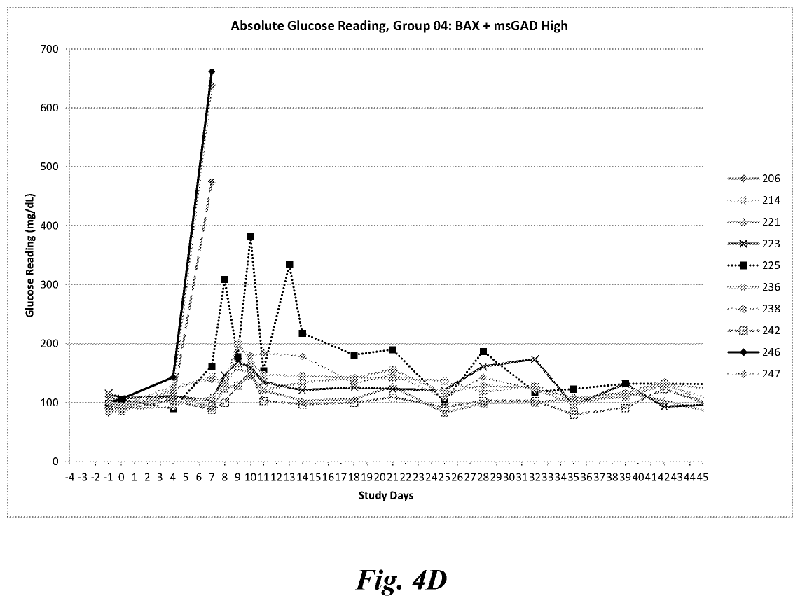

FIG. 3A, FIG. 3B, FIG. 3C, and FIG. 3D (log glucose readings) and FIG. 4A, FIG. 4B, FIG. 4C, and FIG. 4D (absolute glucose readings) present a 45-day observation period and the findings track those of Example 1, FIG. 2A, FIG. 2B, FIG. 2C, and FIG. 2D. It is noted that the three-digit mouse subject identifiers in FIG. 2A, FIG. 2B, FIG. 2C, and FIG. 2D start with the number ‘2’ and that these same mice are again identified in FIG. 3A, FIG. 3B, FIG. 3C, and FIG. 3D and in FIG. 4A, FIG. 4B, FIG. 4C, and FIG. 4D with three-digit mouse subject identifiers that instead of ‘2’ start with the number ‘0’.

FIG. 3A, FIG. 3B, FIG. 3C, and FIG. 3D and FIG. 4A, FIG. 4B, FIG. 4C, and FIG. 4D demonstrate that mice showing normal glycemic levels at 25-days of study continue to show normal glycemic levels for a period of at least 45-days, or for at least about six weeks. That is, no mice showing normal glycemic levels at 25-days subsequently experienced relapse indicated by a BG measurement greater than 200 mg/dL within 45-days of study. These findings add to the information learned from Example 1, such that no mouse in either Example 1 or Example 2 showing normal glycemic levels at 14-days subsequently experienced relapse indicated by a BG measurement greater than 200 mg/dL within 45-days of study.

It is noted that dosing of test articles was paused on D29 after the 5th dose of test articles and ongoing monitoring of blood glucose (BG) was performed. While the study design provided that, in the event of a relapse (i.e., a mouse showing normal glycemic levels at 25-days subsequently having a BG measurement greater than 250 mg/dL), three (3) additional weekly test article doses would be administered, no such relapses occurred within this 45-day study period. These data indicate that the test articles had effectiveness for a period of at least two weeks following the last administration on study day 28, demonstrating a longer term effectiveness of ADI-100 on normalizing blood glucose levels without further administration of test article doses.

Animals from Vehicle Group 2 and low dose BAX+msGAD treatment Group 3 were collected on day 84 (D84) and final measurements and tissue collections were made.

### Example 3

The study described in Example 1 and Example 2 was extended beyond the original study termination date of D84 for animals in control Group 1 and the high dose ADI-100 BAX+msGAD treatment Group 4. This extension was to determine durability of treatment, including preventative treatment, beyond the point when ADI-100 was being administered and whether animals from Group 4, for which dosing was paused on D29 after the 5th dose of test articles, would relapse and develop hyperglycemia and TID disease at a later stage. Upon relapse, dosing would resume to determine whether the high concentration of the test article can reverse disease progression at this later stage or after relapse has occurred. Resumption of additional ADI-100 administration would serve as TID treatment after the onset of disease as seen with the appearance of hyperglycemia suggesting a break in tolerance.

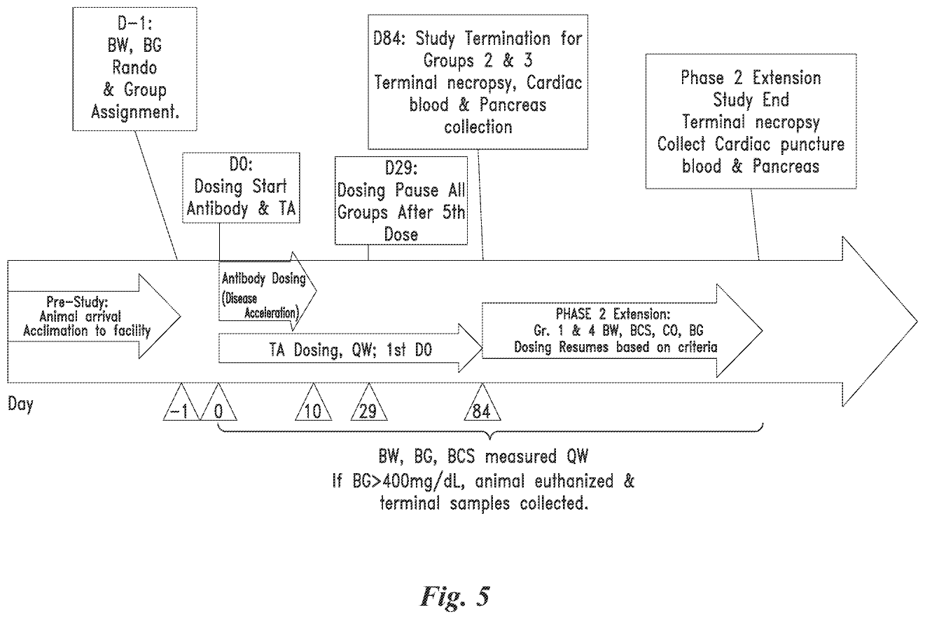

FIG. 5 shows the study design and time course. Remaining mice from Groups 1 and 4 continued on study, with continued body weight (BW), body condition score (BCS), clinical observations (CO), blood glucose (BG) measurements. When blood glucose (BG) increase to >250 mg/dL was observed in Group 4 mice, dosing was resumed for that/those animal(s). Once resumed, dosing was administered once per week (QW) until a BG measurement <250 mg/dl was observed, followed by three additional QW doses; however, any relapsed mouse with a BG measurement >400 mg/dL was euthanized.

Table 2, below, shows the treatment groups and dosing regimen.

| | | | | | Dosing | Dose | | | |
|---|---|---|---|---|---|---|---|---|---|
| | Group | | | | Frequency | Level | Dose | | Day of |
| Group # | Name | N | Treatment | Route | & Duration | (mg/kg) | Volume | | Collection |
| 1 | Control | 10 (7 | IgG | IP | D 0, D 2, | 500 μg | 150 | μL | To be |
| | | remaining at | Isotype | | D 4, D 6, | (D 0), | | | determined |
| | | study day 84; | Control | | D 8, D 10 | 250 μg | | | |
| | | 3 remaining | | | | (D 2-10) | | | |
| | | at study day | | | | | | | |
| | | 112; 3 | | | | | | | |
| | | remaining at | | | | | | | |
| | | study day | | | | | | | |
| | | 301) | | | | | | | |
| | | | Vehicle | ID | QW, 8 | N/A | 50 | μL | |
| | | | | | weeks; first | | | | |
| | | | | | dose D 0; | | | | |
| | | | | | Dosing | | | | |
| | | | | | paused on | | | | |
| | | | | | D 29 (after | | | | |
| | | | | | 5th dose) | | | | |
| 2 | Vehicle | 10 | Anti-PD-1 | IP | D 0, D 2, | 500 μg | 150 | μL | D 84 |
| | | | | | D 4, D 6, | (D 0), | | | |
| | | | | | D 8, D 10 | 250 μg | | | |
| | | | | | | (D 2-10) | | | |
| | | | Vehicle | ID | QW, 8 | N/A | 50 | μL | |
| | | | | | weeks; first | | | | |
| | | | | | dose D 0; | | | | |
| | | | | | Dosing | | | | |
| | | | | | paused on | | | | |
| | | | | | D 29 (after | | | | |
| | | | | | 5th dose) | | | | |
| 3 | BAX + | 10 | Anti-PD-1 | IP | D 0, D 2, | 500 μg | 150 | μL | D 84 |
| | msGAD | | | | D 4, D 6, | (D 0), | | | |
| | Low | | | | D 8, D 10 | 250 μg | | | |
| | | | | | | (D 2-10) | | | |
| | | | BAX 17 | ID | QW, 8 | BAX 17 | 50 | μL | |
| | | | μg + | | weeks; first | μg + | | | |
| | | | msGAD | | dose D 0; | msGAD | | | |
| | | | 33 μg | | Dosing | 33 μg | | | |
| | | | 1 mg/ml | | paused on | | | | |
| | | | | | D 29 (after | | | | |
| | | | | | 5th dose) | | | | |
| 4 | BAX + | 10 (7 | Anti-PD-1 | IP | D 0, D 2, | 500 μg | 150 | μL | To be |
| | msGAD | remaining at | | | D 4, D 6, | (D 0), | | | determined |
| | High | study day | | | D 8, D 10 | 250 μg | | | |
| | | 84; 6 | | | | (D 2-10) | | | |
| | | remaining at | | | | | | | |
| | | study day | | | | | | | |
| | | 301) | | | | | | | |
| | | | BAX 34 | ID | QW, 8 | BAX 34 | 50 | μL | |
| | | | μg + | | weeks; first | μg + | | | |
| | | | msGAD | | dose D 0; | msGAD | | | |
| | | | 66 μg Lot | | Dosing | 66 μg | | | |
| | | | MP52 | | paused on | | | | |
| | | | (155382 + | | D 29 (after | | | | |
| | | | 138766) | | 5th dose) | | | | |
| | | | 2 mg/ml | | | | | | |

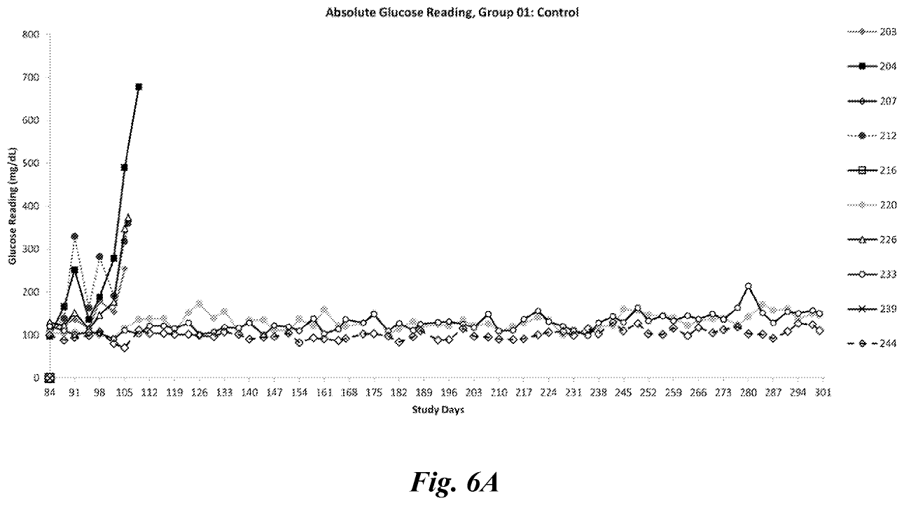

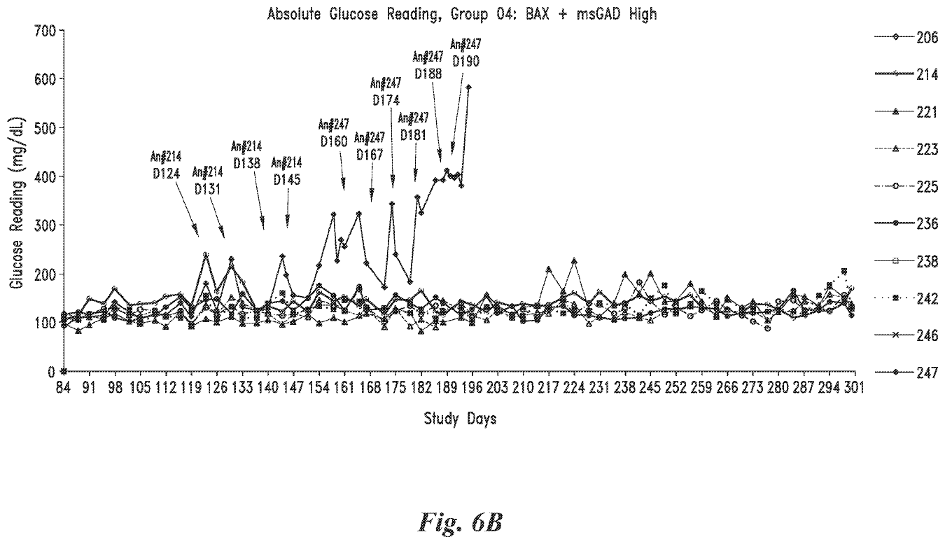

FIG. 6A shows the Absolute Glucose Readings for the Group 1 control mice. It is noted that only three out of the original 10 mice in control Group 1 survived past study day 112 and through study day 301. By contrast, FIG. 6B demonstrates that six out of the original 10 mice in the high-dose Group 4 survived through study day 301. Further, FIG. 6B demonstrates that 6 out of the original 10 mice experienced long-term normalization of blood glucose levels through 301 study days following the last administration of the high dose test article on study day 28. These results indicate the enduring treatment of high blood glucose levels and prevention of TID onset in the ADI-100 high dose Group 4 mice.

As shown in FIG. 6B, two mice, mouse 214 and mouse 247, had relapses where their measured blood glucose (BG) increased to >250 mg/dL. The arrows in FIG. 6B represent each treatment. Mouse 214 relapsed on study day 124, or 96 study days following the last administration of the test article, which was on study day 28. Mouse 214 responded to treatment, was treated four (4) times, and became normal without any further relapse. Mouse 247 relapsed on study day 160, or 132 days following the last administration of the test article, which was on study day 28. Mouse 247 was treated (see arrows) but did not respond to treatment and could not be rescued and succumbed to the natural course of disease. Accordingly, dosing of the high dose test article was resumed as described above. The relapse in mouse 214 was successfully reversed following resumption of the high dose test article administration demonstrating treatment of hyperglycemia after disease occurrence. The relapse in mouse 247 on study day 160 was successfully treated for a period of time, however, mouse 247 again relapsed 36 days later, on study day 196, and was euthanized in accordance with the study protocol.

This study has been conducted in NOD mice, which are prone to develop diabetes. Here, the rate and timing of disease progression were enhanced by applying a checkpoint inhibitor. The manifestation of the disease induced by the checkpoint inhibitor was inhibited in most animals receiving the ADI-100 test article, with the strongest effects demonstrated in the Group 4 high dose test article mice. A second underlying pathology, however, apart from the enhanced diabetes development process induced by a checkpoint inhibitor, which is the natural course driving the NOD mice into the disease, also did not appear to evolve during the further observation time. Thus, there was no further disease development in the remaining healthy animals apart from one of the two relapsed animals which developed high blood glucose levels later in the study after a successful temporary reversal and suppression of elevated blood glucose levels. Accordingly, the data show prevention of disease progression first when treating with a check point inhibitor and second by suppressing the natural course of disease development.

### Example 4

The goal of this study was to test the anti-tumor efficacy of vehicle alone, anti-PD-1 alone, and anti-PD-1 in conjunction with the ADI-100 test article (BAX 34 μg+msGAD66 μg). Anti-PD-1 is reported to be highly efficacious in the Hepa1-6 mouse tumor model.

ADI-100 was supplied by Aditxt, Inc., in a Tris-EDTA (TE) buffer vehicle in frozen vials. One ADI-100 vial per dosing day was thawed overnight at 4° C. before the day of dosing. The Anti-PD-1 in a phosphate-buffered saline (PBS) buffer vehicle was supplied by Bio X Cell, Inc. (catalog #BP0146).

This study was run in the Hepa1-6 liver model using C57BL/6 mice. 30 mice were enrolled in the study. All animals were randomly allocated to the 3 different study groups. Randomization was performed in the Study Log software on day 1. Table 3, below, shows the treatment groups and dosing regimen.

| | | | Dose | Dosing Frequency | Dose | | Dose | |
|---|---|---|---|---|---|---|---|---|
| Group | N | Treatment | Route | & Duration | Level | | Volume | |
| 1 | 10 | Vehicle | I.D. | QW × 6 weeks | N/A | | 50 | μL |
| 2 | 10 | Anti-PD-1 | I.P. | BIW × 6 weeks | 10 | mg/kg | 10 | mL/kg |
| 3 | 10 | Anti-PD-1 | I.P. | BIW × 6 weeks | 10 | mg/kg | 10 | mL/kg |
| | | ADI-100 | I.D. | QW × 6 weeks | 100 | μg | 50 | μL |

Average tumor volume (mm³) for each group+SD at randomization is provided in Table 4 and was as follows:

| | Mean | StdDev | Min | Max |
|---|---|---|---|---|
| Group | (mm³) | (mm³) | (mm³) | (mm³) |
| Group 1: Vehicle | 20.27 | 1.36 | 17.30 | 21.90 |
| Group 2: Anti-PD-1 | 20.57 | 1.05 | 19.20 | 22.50 |
| Group 3: ADI-100 + Anti-PD-1 | 20.48 | 1.46 | 17.80 | 22.70 |

Animals were dosed on study day 1, 4, 8, and 11. The study reference days are indicated in Table 5 below. But the study was terminated following the 4th dose (day 11) due to the rapid treatment result from anti-PD-1.

| | Study Day |
|---|---|
| Study Start | −19 |
| Dosing Start | 1 |
| Dosing End | 11 |
| End of Study | 14 |

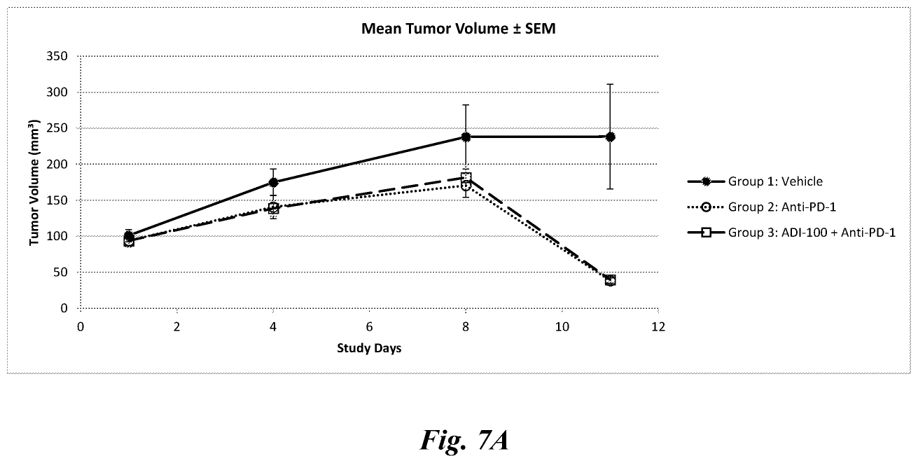

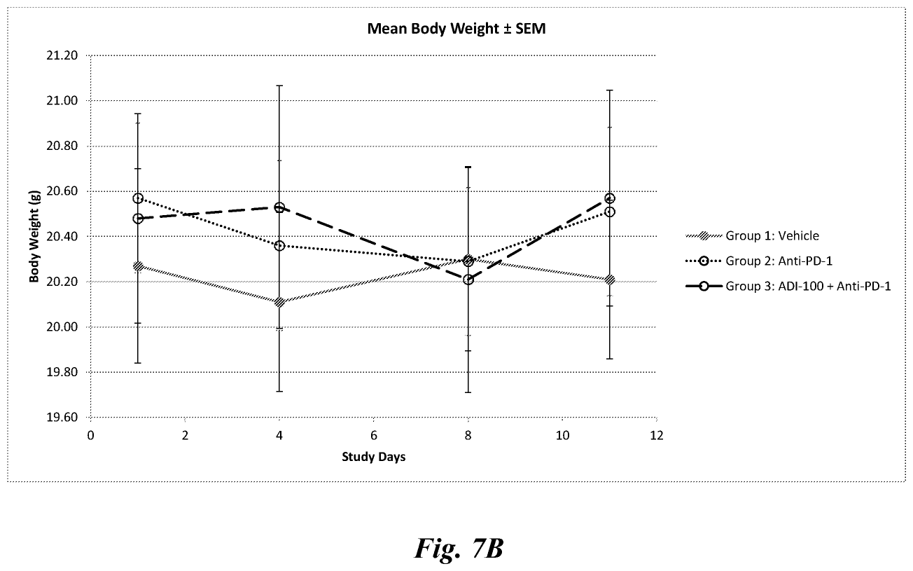

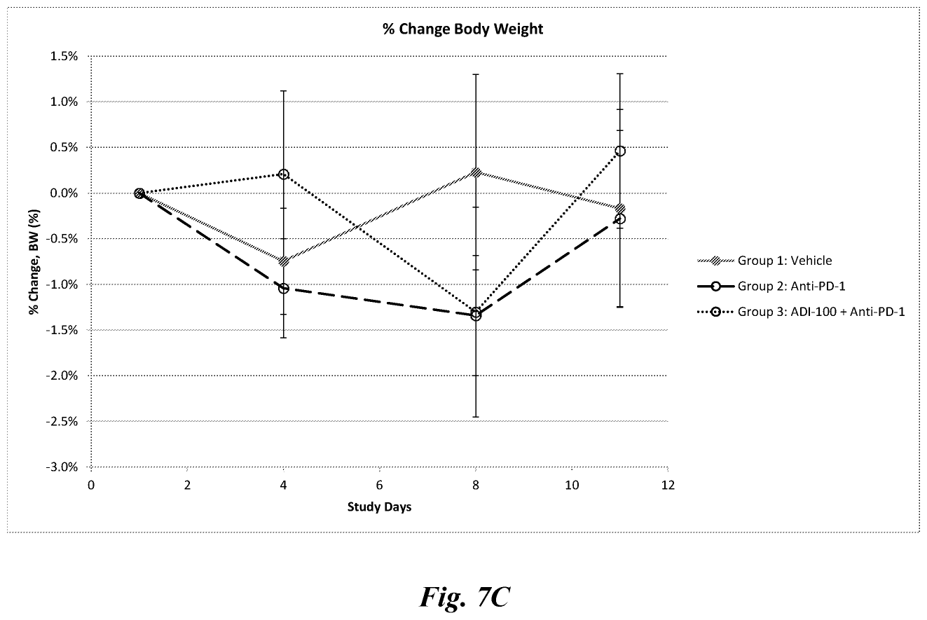

Results from this study are provided in FIG. 7A, FIG. 7B, and FIG. 7C.

As shown in FIG. 7A, dosing started and by day 11, both Group 2 (anti-PD-1 alone) and Group 3 (anti-PD-1+ADI-100) had >80% tumor inhibition compared to the control Group 1 (vehicle alone). And ADI-100 in Group 3 (anti-PD-1+ADI-100) did not negatively interfere with the efficacy displayed by anti-PD-1 compared to Group 2 (anti-PD-1 alone).

FIG. 7B and FIG. 7C show the mean absolute body weight±SEM (standard error of the mean) and mean percent change in body weight±SEM (standard error of the mean), respectively, for each of Group 1, Group 2, and Group 3. ADI-100 did not show any signs of toxicity.

### Example 5

In this study, a syngeneic mouse model will be used to assess the performance of the therapies disclosed herein in an animal model that has a complete and functional immune system. Immune profiling of Tregs, including antigen-specific Tregs, will be performed.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including but not limited to U.S. Provisional Patent Application No. 63/495,984, filed on Apr. 13, 2023, are incorporated herein by reference, in their entirety. Aspects of the embodiments can be modified, if necessary to employ concepts of the various patents, applications and publications to provide yet further embodiments.

These and other changes can be made to the embodiments in light of the above detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A therapeutic composition comprising an immune checkpoint inhibitor and an apoptotic DNA immunotherapy.

2. The therapeutic composition of claim 1, wherein the immune checkpoint inhibitor targets at least one of PD-1 and CTLA-4.

3. The therapeutic composition of claim 1, wherein the apoptotic DNA immunotherapy comprises a polynucleotide encoding BCL2 associated X apoptosis regulator (BAX).

4. The therapeutic composition of claim 1, wherein the apoptotic DNA immunotherapy comprises a polynucleotide encoding BCL2 associated X apoptosis regulator (BAX) and a polynucleotide encoding a secreted form of glutamic acid decarboxylase 65 (GAD65) and, optionally, wherein said polynucleotide encoding a secreted form of glutamic acid decarboxylase 65 (GAD65) is provided as a hypermethylated expression cassette.

5. A method of treating one or more adverse effects resulting from administration of an immune checkpoint inhibitor to a subject in need thereof comprising administration of an apoptotic DNA immunotherapy.

6. The method of claim 5, further comprising administering at least one of an anti-PD-1 immune checkpoint inhibitor and an anti-CTLA-4 immune checkpoint inhibitor.

7. The method of claim 5, further comprising administering an apoptotic DNA immunotherapy comprising a polynucleotide encoding BCL2 associated X apoptosis regulator (BAX).

8. The method of claim 5, further comprising administering an apoptotic DNA immunotherapy which comprises a polynucleotide encoding BCL2 associated X apoptosis regulator (BAX) and a polynucleotide encoding a secreted form of glutamic acid decarboxylase 65 (GAD65) and, optionally, wherein said polynucleotide encoding a secreted form of glutamic acid decarboxylase 65 (GAD65) is provided as a hypermethylated expression cassette.

9. The method of claim 5, wherein development of type 1 diabetes mellitus is prevented or wherein hyperglycemia associated with development of type 1 diabetes mellitus is prevented or reduced.

10. (canceled)

11. The method of claim 5, further comprising one of:
(i) administering the apoptotic DNA immunotherapy at any one or more times before, during, or after administration of an immune checkpoint inhibitor;
(ii) stopping administration of the apoptotic DNA immunotherapy at any one or more times before, during, or after administration of an immune checkpoint inhibitor upon indication that antigen-specific immune tolerance has been achieved in a subject in need thereof;
(iii) maintaining the effectiveness of the immune checkpoint inhibitor; or
(iv) resuming administration of the apoptotic DNA immunotherapy at any one or more times before, during, or after administration of an immune checkpoint inhibitor upon indication that antigen-specific immune tolerance has been lost in a subject in need thereof.

12. (canceled)

13. The method of claim 5, wherein the subject has cancer.

14. The method of claim of 13 wherein tumor growth is inhibited.

15. A method of preventing adverse effects resulting from administration of an immune checkpoint inhibitor to a subject in need thereof comprising administration of an apoptotic DNA immunotherapy.

16. The method of claim 15, further comprising administering at least one of an anti-PD-1 immune checkpoint inhibitor and an anti-CTLA-4 immune checkpoint inhibitor.

17. The method of claim 15, further comprising administering an apoptotic DNA immunotherapy comprising a polynucleotide encoding BCL2 associated X apoptosis regulator (BAX).

18. The method of claim 15, further comprising administering an apoptotic DNA immunotherapy comprises a polynucleotide encoding BCL2 associated X apoptosis regulator (BAX) and a polynucleotide encoding a secreted form of glutamic acid decarboxylase 65 (GAD65) and, optionally, wherein said polynucleotide encoding a secreted form of glutamic acid decarboxylase 65 (GAD65) is provided as a hypermethylated expression cassette.

19. The method of claim 15, wherein development of type 1 diabetes mellitus is prevented, or wherein hyperglycemia associated with development of type 1 diabetes mellitus is prevented or reduced.

20. (canceled)

21. The method of claim 15, further comprising one of:
(i) administering the apoptotic DNA immunotherapy at any one or more times before, during, or after administration of an immune checkpoint inhibitor;
(ii) stopping administration of the apoptotic DNA immunotherapy at any one or more times before, during, or after administration of an immune checkpoint inhibitor upon indication that antigen-specific immune tolerance has been achieved in a subject in need thereof;
(iii) maintaining the effectiveness of immune checkpoint inhibitor; or
(iv) resuming administration of the apoptotic DNA immunotherapy at any one or more times before, during, or after administration of an immune checkpoint inhibitor upon indication that antigen-specific immune tolerance has been lost in a subject in need thereof.

22-28. (canceled)

29. The method of claim 15, wherein the subject has cancer and, optionally, wherein tumor growth is inhibited.

30-36. (canceled)
